SEQUENCE LISTING (6 sequences)

SEQ ID NO: 1 - DNA, 5912 bp, Unknown
  Description of Unknown: Notch 4 sequence
atgcagcccccttcactgctgctgctgctgctgctgctgctgctgctatgtgtctcagtggtcagacccagagggctgctgtgtgggagtttcccagaaccctgtgccaatggaggcacctgcctgagcctgtctctgggacaagggacctgccagtgtgcccctggcttcctgggtgagacgtgccagtttcctgacccctgccagaacgcccagctctgccaaaatggaggcagctgccaagccctgcttcccgctcccctagggctccccagctctccctctccattgacacccagcttcttgtgcacttgcctccctggcttcactggtgagagatgccaggccaagcttgaagacccttgtcctccctccttctgttccaaaaggggccgctgccacatccaggcctcgggccgcccacagtgctcctgcatgcctggatggacaggtgagcagtgccagcttcgggacttctgttcagccaacccatgtgttaatggaggggtgtgtctggccacatacccccagatccagtgccactgcccaccgggcttcgagggccatgcctgtgaacgtgatgtcaacgagtgcttccaggacccaggaccctgccccaaaggcacctcctgccataacaccctgggctccttccagtgcctctgccctgtggggcaggagggtccacgttgtgagctgcgggcaggaccctgccctcctaggggctgttcgaatgggggcacctgccagctgatgccagagaaagactccacctttcacctctgcctctgtcccccaggtttcataggcccagactgtgaggtgaatccagacaactgtgtcagccaccagtgtcagaatgggggcacttgccaggatgggctggacacctacacctgcctctgcccagaaacctggacaggctgggactgctccgaagatgtggatgagtgtgagacccagggtccccctcactgcagaaacgggggcacctgccagaactctgctggtagctttcactgcgtgtgtgtgagtggctggggcggcacaagctgtgaggagaacctggatgactgtattgctgccacctgtgccccgggatccacctgcattgaccgggtgggctctttctcctgcctctgcccacctggacgcacaggactcctgtgccacttggaagacatgtgtctgagccagccgtgccatggggatgcccaatgcagcaccaaccccctcacaggctccacactctgcctgtgtcagcctggctattcggggcccacctgccaccaggacctggacgagtgtctgatggcccagcaaggcccaagtccctgtgaacatggcggttcctgcctcaacactcctggctccttcaactgcctctgtccacctggctacacaggctcccgttgtgaggctgatcacaatgagtgcctctcccagccctgccacccaggaagcacctgtctggacctacttgccaccttccactgcctctgcccgccaggcttagaagggcagctctgtgaggtggagaccaacgagtgtgcctcagctccctgcctgaaccacgcggattgccatgacctgctcaacggcttccagtgcatctgcctgcctggattctccggcacccgatgtgaggaggatatcgatgagtgcagaagctctccctgtgccaatggtgggcagtgccaggaccagcctggagccttccactgcaagtgtctcccaggctttgaagggccacgctgtcaaacagaggtggatgagtgcctgagtgacccatgtcccgttggagccagctgccttgatcttccaggagccttcttttgcctctgcccctctggtttcacaggccagctctgtgaggttcccctgtgtgctcccaacctgtgccagcccaagcagatatgtaaggaccagaaagacaaggccaactgcctctgtcctgatggaagccctggctgtgccccacctgaggacaactgcacctgccaccacgggcactgccagagatcctcatgtgtgtgtgacgtgggttggacggggccagagtgtgaggcagagctagggggctgcatctctgcaccctgtgcccatggggggacctgctacccccagccctctggctacaactgcacctgccctacaggctacacaggacccacctgtagtgaggagatgacagcttgtcactcagggccatgtctcaatggcggctcctgcaaccctagccctggaggctactactgcacctgccctccaagccacacagggccccagtgccaaaccagcactgactactgtgtgtctgccccgtgcttcaatgggggtacctgtgtgaacaggcctggcaccttctcctgcctctgtgccatgggcttccagggcccgcgctgtgagggaaagctccgccccagctgtgcagacagcccctgtaggaatagggcaacctgccaggacagccctcagggtccccgctgcctctgccccactggctacaccggaggcagctgccagactctgatggacttatgtgcccagaagccctgcccacgcaattcccactgcctccagactgggccctccttccactgcttgtgcctccagggatggaccgggcctctctgcaaccttccactgtcctcctgccagaaggctgcactgagccaaggcatagacgtctcttccctttgccacaatggaggcctctgtgtcgacagcggcccctcctatttctgccactgcccccctggattccaaggcagcctgtgccaggatcacgtgaacccatgtgagtccaggccttgccagaacggggccacctgcatggcccagcccagtgggtatctctgccagtgtgccccaggctacgatggacagaactgctcaaaggaactcgatgcttgtcagtcccaaccctgtcacaaccatggaacctgtactcccaaacctggaggattccactgtgcctgccctccaggctttgtggggctacgctgtgagggagacgtggacgagtgtctggaccagccctgccaccccacaggcactgcagcctgccactctctggccaatgccttctactgccagtgtctgcctggacacacaggccagtggtgtgaggtggagatagacccctgccacagccaaccctgctttcatggagggacctgtgaggccacagcaggatcacccctgggtttcatctgccactgccccaagggttttgaaggccccacctgcagccacagggccccttcctgcggcttccatcactgccaccacggaggcctgtgtctgccctcccctaagccaggcttcccaccacgctgtgcctgcctcagtggctatgggggtcctgactgcctgaccccaccagctcctaaaggctgtggccctccctccccatgcctatacaatggcagctgctcagagaccacgggcttggggggcccaggctttcgatgctcctgccctcacagctctccagggccccggtgtcagaaacccggagccaaggggtgtgagggcagaagtggagatggggcctgcgatgctggctgcagtggcccgggaggaaactgggatggaggggactgctctctgggagtcccagacccctggaagggctgcccctcccactctcggtgctggcttctcttccgggacgggcagtgccacccacagtgtgactctgaagagtgtctgtttgatggctacgactgtgagacccctccagcctgcactccagcctatgaccagtactgccatgatcacttccacaacgggcactgtgagaaaggctgcaacactgcagagtgtggctgggatggaggtgactgcaggcctgaagatggggacccagagtgggggccctccctggccctgctggtggtactgagccccccagccctagaccagcagctgtttgccctggcccgggtgctgtccctgactctgagggtaggactctgggtaaggaaggatcgtgatggcagggacatggtgtacccctatcctggggcccgggctgaagaaaagctaggaggaactcgggaccccacctatcaggagagagcagcccctcaaacgcagcccctgggcaaggagaccgactccctcagtgctgggtttgtggtggtcatgggtgtggatttgtcccgctgtggccctgaccacccggcatcccgctgtccctgggaccctgggcttctactccgcttccttgctgcgatggctgcagtgggagccctggagcccctgctgcctggaccactgctggctgtccaccctcatgcagggaccgcaccccctgccaaccagcttccctggcctgtgctgtgctccccagtggccggggtgattctcctggccctaggggctcttctcgtcctccagctcatccggcgtcgacgccgagagcatggagctctctggctgccccctggtttcactcgacggcctcggactcagtcagctccccaccgacgccggcccccactaggcgaggacagcattggtctcaaggcactgaagccaaaggcagaagttgatgaggatggagttgtgatgtgctcaggccctgaggagggagaggaggtgggccaggctgaagaaacaggcccaccctccacgtgccagctctggtctctgagtggtggctgtggggcgctccctcaggcagccatgctaactcctccccaggaatctgagatggaagcccctgacctggacacccgtggacctgatggggtgacacccctgatgtcagcagtttgctgtggggaagtacagtccgggaccttccaaggggcatggttgggatgtcctgagccctgggaacctctgctggatggaggggcctgtccccaggctcacaccgtgggcactggggagacccccctgcacctggctgcccgattctcccggccaaccgctgcccgccgcctccttgaggctggagccaaccccaaccagccagaccgggcagggcgcacaccccttcatgctgctgtggctgctgatgctcgggaggtctgccagcttctgctccgtagcagacaaactgcagtggacgctcgcacagaggacgggaccacacccttgatgctggctgccaggctggcggtggaagacctggttgaagaactgattgcagcccaagcagacgtgggggccagagataaatgggggaaaactgcgctgcactgggctgctgccgtgaacaacgcccgagccgcccgctcgcttctccaggccggagccgataaagatgcccaggacaacagggagcagacgccgctattcctggcggcgcgggaaggagcggtggaagtagcccagctactgctggggctgggggcagcccgagagctgcgggaccaggctgggctagcgccggcggacgtcgctcaccaacgtaaccactgggatctgctgacgctgctggaaggggctgggccaccagaggcccgtcacaaagccacgccgggccgcgaggctgggcccttcccgcgcgcacggacggtgtcagtaagcgtgcccccgcatgggggcggggctctgccgcgctgccggacgctgtcagccggagcaggccctcgtgggggcggagcttgtctgcaggctcggacttggtccgtagacttggctgcgcgggggggcggggcctattctcattgccggagcctctcgggagtaggagcaggaggaggcccgacccctcgcggccgtaggttttctgcaggcatgcgcgggcctcggcccaaccctgcgataatgcgaggaagatacggagtggctgccgggcgcggaggcagggtctcaacggatgactggccctgtgattgggtggccctgggagcttgcggttctgcctccaacattccgatcccgcctccttg

SEQ ID NO: 2 - protein, 2003 aa, Unknown
  Description of Unknown: Notch 4 sequence
MQPPSLLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEVGQAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 3 - protein, 115 aa, Artificial Sequence
  Description of Artificial Sequence: Synthetic polypeptide
EVQLVESGGGLVQPGGSLRLSCAASGFTFSSYGMSWVRQAPGKGLELVATINSNGGRTYYPDSVKGRFTISRDNSKNTLYLQMGSLKAEDMAVYYCARDQGFAYWGQGTLVTVSS

SEQ ID NO: 4 - protein, 107 aa, Artificial Sequence
  Description of Artificial Sequence: Synthetic polypeptide
EIVMTQSPATLSVSPGERATLSCKASQDVGTAVAWYQQKPGQAPRLLIYWASTRHTGIPARFSGSGSGTEFTLTISSLQSEDFAVYYCQQYSSYPWTFGQGTKVEIK

SEQ ID NO: 5 - protein, 123 aa, Artificial Sequence
  Description of Artificial Sequence: Synthetic polypeptide
QVQLVESGGGVVQPGRSLRLSCAASGFTFSSYGMHWVRQAPGEGLEWVATIPASGDNTYYADSVEGRFTISRDNSENTLYLQMNSLRAEDTAVYYCAKGAYKGYYYYIWWDVWGQGTMVTVSS

SEQ ID NO: 6 - protein, 110 aa, Artificial Sequence
  Description of Artificial Sequence: Synthetic polypeptide
QSALTQPASVSGSPGQSITISCTGTSSDVGGYNYVSWYQQHPGEAPKLMIYEVSKRPSGVSNRFSGSKSGNTASLTISGLQAEDEADYYCSSYTRRSQRVFGGGTKLTVL

(19) United States
(12) Patent Application Publication
(10) Pub. No.: US 2023/0226095 A1
(43) Pub. Date: Jul. 20, 2023

(54) METHODS AND COMPOSITIONS FOR TREATING CORONAVIRUS INFECTIOUS DISEASE

(71) Applicant: The Children's Medical Center Corporation, Boston, MA (US)

(72) Inventors: Talal Chatila, Belmont, MA (US); Hani Harb, Brookline, MA (US); Mehdi Benamar, Boston, MA (US)

(73) Assignee: The Children's Medical Center Corporation, Boston, MA (US)

(21) Appl. No.: 18/009,605

(22) PCT Filed: Jun. 11, 2021
(86) PCT No.: PCT/US2021/037009

§ 371 Date: Dec. 9, 2022

Related U.S. Application Data
(60) Provisional application No. 63/038,186, filed on Jun. 12, 2020.

(51) Int. Cl. A61K 31/7125; A61K 31/713; A61K 45/06; A61P 11/00; A61P 31/14

(52) U.S. Cl. CPC A61K 31/7125; A61K 31/713; A61K 45/06; A61P 11/00; A61P 31/14

(57) ABSTRACT

Described herein are methods and compositions for treating a coronavirus infectious disease, e.g., COVID-19. Aspects of the invention relate to administering to a subject an agent that targets Notch4.

### CROSS-REFERENCE TO RELATED APPLICATION

This application is a 35 U.S.C. § 371 National Phase Entry Application of International Patent Application No. PCT/US2021/037009 filed Jun. 11, 2021, which designated the U.S., and which claims the benefit of and priority to under 35 U.S.C. § 119(e) of U.S. Provisional Application No. 63/038,186 filed on Jun. 12, 2020, the contents of which are incorporated herein by reference in their entireties.

### GOVERNMENT SUPPORT

This invention was made with government support under Grant Number AI065617 awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Jul. 21, 2021, is named 701039-097800WOPT_SL .txt and is 30,260 bytes in size.

### BACKGROUND

COVID19, caused by the betacoronavirus (CoV) Glade SARS-CoV-2, ranges from asymptomatic disease to fatal multi-organ failure. COVID-19 disease severity may be influenced by adverse environmental exposures such as to air pollution and underlying comorbidities such as obesity, hypertension and diabetes. Both innate immunity, particularly type I interferons, and T cell-mediated adaptive immunity are important for limiting viral replication. To date, very limited information is available on the immune status of subjects with COVID19. A thorough understanding the mechanisms of immune dysregulation in COVID-19 is crucial for the development of targeted effective therapies.

### SUMMARY

In one aspect, the present invention relates to a method for treating or ameliorating a symptom of a coronavirus infectious disease comprising administering to a subject having a coronavirus infectious disease an effective amount of an agent that inhibits Notch4.

In one embodiment of any aspect, the coronavirus infectious disease is COVID-19.

In one embodiment of any aspect, the method further comprises the step of, prior to administering, diagnosing the subject as having coronavirus infectious disease.

In one embodiment of any aspect, the method further comprises the step of, prior to administering, receiving the results of an assay that diagnoses the subject as having coronavirus infectious disease.

In one embodiment of any aspect, the agent that inhibits Notch4 is selected from the group consisting of a small molecule, an antibody or antigen-binding fragment thereof, a peptide, a genome editing system, an antisense oligonucleotide, and an RNAi.

In one embodiment of any aspect, the antibody or antigen-binding fragment thereof is a humanized antibody or antigen-binding fragment thereof, e.g., a humanized Notch4 antibody or Notch4-binding fragment thereof.

In one embodiment of any aspect, the RNAi is a microRNA, an siRNA, or a shRNA.

In one embodiment of any aspect, inhibiting Notch4 is inhibiting the expression level and/or activity of Notch4.

In one embodiment of any aspect, the expression level and/or activity of Notch4 is inhibited by at least 50%, at least 60%, at least 70%, at least 80%, at least 90%, or more as compared to an appropriate control or reference level.

In one embodiment of any aspect, Notch4 is inhibited on T regulatory cells.

In one embodiment of any aspect, the method further comprises administering at least one additional therapeutic.

In one embodiment of any aspect, the at least one additional therapeutic is an anti-viral therapeutic.

Another aspect provided herein is a method for treating COVID-19 comprising administering to a subject having COVID-19 an effective amount of an agent that inhibits Notch4.

In one embodiment of any aspect, the method further comprises the step of, prior to administering, diagnosing the subject as having COVID-19.

In one embodiment of any aspect, the method further comprises the step of, prior to administering, receiving the results of an assay that diagnoses the subject as having COVID-19.

Another aspect provided herein is a method for preventing a coronavirus infectious disease, comprising administering to a subject at risk of developing a coronavirus infectious disease an agent that inhibits Notch4.

In one embodiment of any aspect, the method further comprises the step of, prior to administering, identifying a subject at risk of developing the coronavirus infectious disease.

In one embodiment of any aspect, the method further comprises the step of, prior to administering, receiving the results of an assay that identifies a subject as being at risk of developing the coronavirus infectious disease.

Another aspect provided herein is a method for preventing COVID-19, comprising administering to a subject at risk of developing a COVID-19 disease an agent that inhibits Notch4.

In one embodiment of any aspect, the method further comprises the step of, prior to administering, identifying a subject at risk of developing COVID-19.

In one embodiment of any aspect, the method further comprises the step of, prior to administering, receiving the results of an assay that identifies a subject as being at risk of developing COVID-19.

Another aspect provided herein is a composition for the treatment of a coronavirus infectious disease, the composition comprising an agent that inhibits Notch4 and a pharmaceutically acceptable carrier.

Another aspect provided herein is a composition for the treatment of a COVID-19, the composition comprising an agent that inhibits Notch4 and a pharmaceutically acceptable carrier.

In one embodiment, the composition is formulated for inhaled administration.

Another aspect provided herein is a method for treating a subject at risk of developing acute respiratory distress syndrome (ARDS) comprising (a) receiving the results of an assay that identifies a subject as being at risk of developing ARDS when the level of Notch4 is increased as compared to a reference level; and (b) administering an agent that inhibits Notch4 to a subject identified as being at risk of developing ARDS.

In one embodiment of any aspect, the subject was diagnosed as having COVID-19 prior to obtaining a biological sample.

In one embodiment of any aspect, the method further comprises the step of, prior to obtaining a biological sample, diagnosing a subject as having COVID-19.

In one embodiment of any aspect, the method further comprises the step of, prior to obtaining a biological sample, receiving the results of an assay that diagnoses a subject as having COVID-19.

In one embodiment of any aspect, before the step of administering, the level of Notch4 in the subject is increased by at least 2-fold, at least 3-fold, at least 4-fold, at least 5-fold, at least 6-fold, at least 7-fold, at least 8-fold, at least 9-fold, at least 10-fold, or more as compared to a reference level.

Another aspect provided herein is a method for treating a subject having acute respiratory distress syndrome (ARDS) comprising (a) receiving the results of an assay that identifies a subject as having ARDS when the level of Notch is increased as compared to a reference level; and (b) administering an agent that inhibits Notch4 to a subject identified as having ARDS.

In one aspect, provided are methods for treating, preventing, or ameliorating a symptom associated with a coronavirus infectious disease, the method comprising administering to a subject having or at risk of having a coronavirus infectious disease an effective amount of a Notch4 modulating agent.

In some embodiments, the coronavirus infectious disease is COVID19.

In some embodiments, the Notch4 modulating agent is selected from the group consisting of a small molecule, an antibody or antigen-binding fragment thereof, a peptide, a genome editing system, an antisense oligonucleotide, and an RNAi.

In some embodiments, the Notch4 modulating agent is a Notch4 antibody or Notch4-binding fragment thereof, e.g., a humanized Notch4 antibody or Notch4-binding fragment thereof.

In some embodiments, the Notch4 modulating agent reduces the expression level and/or activity of Notch4.

In some embodiments, the Notch4 modulating agent reduces the expression level and/or activity of Notch4 by at least 50%, at least 60%, at least 70%, at least 80%, at least 90%, or more as compared to an appropriate control or reference level.

In one aspect, provided are compositions for the treatment, prevention, or amelioration of a symptom associated with coronavirus infectious disease. In some embodiments, a composition comprises a Notch4 modulating agent and a pharmaceutically acceptable carrier.

In one aspect, provided a method of treating or preventing acute respiratory distress syndrome (ARDS), in a subject in need thereof, the method comprising, administering to the subject an effective amount of an agent that modulates Notch4.

In some embodiments, the agent that modulates Notch4 is selected from the group consisting of a small molecule, an antibody or antigen-binding fragment thereof, a peptide, a genome editing system, an antisense oligonucleotide, and an agent that is an RNA interfering (RNAi) agent.

In some embodiments, the antibody or antigen-binding fragment thereof is a humanized antibody or antigen-binding fragment thereof. For example, the humanized antibody or antigen-binding fragment thereof may be a humanized Notch4 antibody or antigen-binding fragment thereof.

In some embodiments, the RNAi agent is a microRNA, an siRNA, or a shRNA.

In some embodiments, Notch4 is modulated by reducing the expression level and/or activity of Notch4. In some embodiments, the expression level and/or activity of Notch4 is reduced by at least 50%, at least 60%, at least 70%, at least 80%, at least 90%, or more as compared to an appropriate control or reference level.

In some embodiments, the agent modulates Notch4 by inhibiting expression and/or activity of Notch4.

In some embodiments, provided are methods of treating or preventing acute respiratory distress syndrome (ARDS), in a subject in need thereof, the method comprising, administering to the subject an effective amount of an antibody or antigen-binding fragment thereof that modulates Notch4 activity.

In some embodiments, the antibody or antigen-binding fragment thereof is a humanized antibody, a chimeric antibody, a nanobody, an affibody, an scFv, an Fab, or an antigen-binding fragment thereof.

In some embodiments, Notch4 is modulated on T regulatory cells.

In some embodiments, provided methods further comprise administering at least one additional therapeutic. For example, the additional therapeutic may be an anti-viral therapeutic.

In some embodiments, the level of Notch4 in the subject before the step of administering is increased by at least 2-fold, at least 3-fold, at least 4-fold, at least 5-fold, at least 6-fold, at least 7-fold, at least 8-fold, at least 9-fold, at least 10-fold, or more as compared to a reference level.

In some embodiments, the level of Notch4 in the subject is determined before administration.

In some embodiments, the level of Notch4 in the subject is monitored after administration of the agent.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

As used herein, the terms “about” and “approximately” are used interchangeably. When used herein in reference to a value, “about” or “approximately” refers to a value that is similar in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the term “antibody” refers to a polypeptide whose amino acid sequence includes immunoglobulins and fragments thereof which specifically bind to a designated antigen, or fragments thereof. Unless otherwise specified, antibodies may be of any type (e.g., IgA, IgD, IgE, IgG, or IgM) or subtype (e.g., IgA1, IgA2, IgG1, IgG2, IgG3, or IgG4). Those of ordinary skill in the art will appreciate that a characteristic sequence or portion of an antibody may include amino acids found in one or more regions of an antibody (e.g., variable region, hypervariable region, constant region, heavy chain, light chain, and combinations thereof). Moreover, those of ordinary skill in the art will appreciate that a characteristic sequence or portion of an antibody may include one or more polypeptide chains, and may include sequence elements found in the same polypeptide chain or in different polypeptide chains. In some embodiments, antibodies are humanized or chimeric.

As used herein, “antigen-binding fragment” refers to a portion of an antibody that retains the binding characteristics of the parent antibody. In some embodiments, antigen-binding fragments are selected from nanobodies, affibodies, scFvs, and Fabs.

As used herein, the terms “treat,” “treatment,” “treating,” or “amelioration” refer to therapeutic treatments, wherein the object is to reverse, alleviate, ameliorate, inhibit, slow down or stop the progression or severity of a condition or symptom associated with a coronavirus infectious disease (e.g., COVID-19 or acute respiratory distress syndrome (ARDS)). In the context of a particular disease, the term “treating” includes reducing or alleviating at least one adverse effect or symptom of the disease (e.g. COVID-19 (e.g., a symptom such as difficulty breathing), ARDS, etc.). Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein “preventing” or “prevention” refers to any methodology where the disease state or disorder (e.g., a coronavirus infectious disease (such as COVID-19) or ARDS) does not occur due to the actions of the methodology (such as, for example, administration of a Notch4 modulating agent (e.g., an agent that inhibits Notch4), or a composition described herein). In one aspect, it is understood that prevention can also mean that the disease is not established to the extent that occurs in untreated controls. For example, there can be a 5, 10, 15, 20, 25, 30, 35, 40, 50, 60, 70, 80, 90, or 100% reduction in the establishment of disease frequency relative to untreated controls. Accordingly, prevention of a disease encompasses a reduction in the likelihood that a subject will develop the disease, relative to an untreated subject (e.g. a subject who is not treated with a composition comprising an agent described herein).

As used herein, the term “administering,” refers to the placement of a therapeutic (e.g., a Notch4 modulating agent (e.g., an agent that inhibits Notch4)) or composition as disclosed herein into a subject by a method or route which results in at least partial delivery of the agent to the subject. Pharmaceutical compositions comprising agents as disclosed herein can be administered by any appropriate route which results in an effective treatment in the subject.

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, bat, horse, or cow, but is not limited to these examples. Mammals other than humans can be advantageously used as subjects that represent animal models of disease e.g., coronavirus infectious disease. A subject can be male or female. A subject can be a child (e.g., less than 18 years of age), or an adult (e.g., greater than 18 years of age).

A subject can be one who has been previously diagnosed with or identified as suffering from or having a disease or disorder in need of treatment (e.g., coronavirus infectious disease (such as COVID-19) or ARDS) or one or more complications related to such a disease or disorder, and optionally, have already undergone treatment for the disease or disorder or the one or more complications related to the disease or disorder. Alternatively, a subject can also be one who has not been previously diagnosed as having such disease or disorder (e.g., COVID-19 or ARDS) or related complications. For example, a subject can be one who exhibits one or more risk factors for the disease or disorder or one or more complications related to the disease or disorder or a subject who does not exhibit risk factors.

As used herein, an “agent” that modulates or inhibits a particular target refers to e.g., a molecule, protein, peptide, antibody, or nucleic acid, that modulates or inhibits expression of that target, e.g., a polypeptide or polynucleotide, or binds to, partially or totally blocks stimulation, decreases, prevents, delays activation, inactivates, desensitizes, or down regulates the activity of the target, e.g., a polypeptide or the polynucleotide. Agents that modulate or inhibit Notch4, e.g., modulate or inhibit expression, e.g., translation, post-translational processing, stability, degradation, or nuclear or cytoplasmic localization of a polypeptide, or transcription, post transcriptional processing, stability or degradation of a polynucleotide or bind to, partially or totally block stimulation, DNA binding, transcription factor activity or enzymatic activity, decrease, prevent, delay activation, inactivate, desensitize, or down regulate the activity of a polypeptide or polynucleotide. An agent can act directly or indirectly.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “COVID-19” as used herein refers to a respiratory infection caused by the coronavirus SARS-CoV-2.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNA, shRNA, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

In some embodiments, methods and compositions described herein are characterized in that the levels and/or activity of Notch4 are modulated or inhibited. As used herein, Neurogenic locus notch homolog 4, also known as “Notch4” refers to a type I transmembrane protein, which is a member of a family that share structural characteristics, including an extracellular domain consisting of multiple epidermal growth factor-like (EGF) repeats, and an intracellular domain consisting of multiple different domain. Notch4 sequences are known for a number of species, e.g., human Notch4 (NCBI Gene ID: 4855) polypeptide (e.g., NCBI Ref Seq NP_004548.3) and mRNA (e.g., NCBI Ref Seq NM_004557.3). Notch4 can refer to human Notch4, including naturally occurring variants, molecules, and alleles thereof. Notch4 refers to the mammalian Notch4 of, e.g., mouse, rat, rabbit, dog, cat, cow, horse, pig, and the like. The nucleic sequence of SEQ ID NO: 1 comprises a nucleic sequence which encodes Notch4.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to an appropriate control (e.g. the absence of a given treatment) or reference level and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “partial reduction” or “partial inhibition” does not encompass a complete inhibition or reduction as compared to a reference level. “Complete inhibition” is a 100% inhibition as compared to an appropriate control or reference level.

The terms “increase”, “enhance”, or “activate” are all used herein to mean an increase by a reproducible statistically significant amount. In some embodiments, the terms “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to an appropriate control or reference level. In the context of a marker, an “increase” is a reproducible statistically significant increase in such level.

As used herein, a “reference level” refers to the level of refers to the level observed under appropriate reference conditions. For example, in some embodiments, the reference level is a level as determined by the use of said method with a control in an experimental animal model or clinical trial. In some embodiments, the reference level is a level in the same subject before or at the beginning of treatment. In some embodiments, the reference level is the average level in a population not being treated by said method of treatment. In some embodiments, the reference level refers to the level in a normal, otherwise unaffected cell population or tissue (e.g., a biological sample obtained from a healthy subject, or a biological sample obtained from the subject at a prior time point, e.g., a biological sample obtained from a patient prior to being diagnosed with a coronavirus infectious disease, or a biological sample that has not been contacted with an agent disclosed herein), or subject.

As used herein, an “appropriate control” refers to an untreated, otherwise identical cell or population (e.g., a patient who was not administered an agent described herein, or was administered by only a subset of agents described herein, as compared to a non-control cell), or subject.

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIGS. 1A-1G show increased expression of Notch4 on circulating Treg cells of COVID-19 subjects. FIGS. 1A-1D, Flow cytometric analysis, cell frequencies, absolute numbers and MFI of Notch4 expression in Treg cells (FIGS. 1A, 1B) and Teff cells (FIGS. 1C, 1D) of control subjects and patients with mild, moderate, severe or resolved COVID-19 (healthy controls n=37, mild patients n=20, moderate patients n=54, severe patients n=36, and convalescent subjects n=6). FIG. 1E, Serum concentrations of IL-6 in the different subject groups (healthy controls n=37, mild patients n=18, moderate patients n=45, and severe patients n=21). FIG. 1F, Correlation analysis of Notch4 expression on Treg cells of patient and control subjects as a function of serum IL-6 concentrations (n=121). FIG. 1G, serum concentrations of IFNa, IFN13, IFN1 IFNA, CXCL10, IL-113, IL-8, IL-10, IL-12 and TNF in control and patient subjects (healthy controls n=37, mild patients n=18, moderate patients n=45, and severe patients n=21). Each symbol represents one subject. Numbers in flow plots indicate percentages. Error bars indicate SEM. Statistical tests: *P<0.05, **P<0.01, ****P<0.0001 by one-way ANOVA with Dunnett’s post hoc analysis (FIGS. 1A-1E; 1G) and Pearson correlation analysis (FIG. 1D). Data representative of two or three independent experiments.

FIGS. 2A-2C show expression of Notch1, Notch2 and Notch3 on circulating Treg cells of control and COVID-19 subjects. FIGS. 2A-2C Flow cytometric analysis and graphical representation of Notch1 (FIG. 2A), Notch2 (FIG. 2B) and Notch3 (FIG. 2C) expression in Treg cells of control and COVID-19 subject groups. Each symbol represents one subject. Numbers in flow plots indicate percentages. Error bars indicate SEM. Statistical tests: One-way ANOVA with Dunnett’s post hoc analysis.

FIGS. 3A-3F show protective effect of Notch4 deletion in poly I:C-induced lung injury. FIGS. 3A-3B flow cytometric analysis (FIG. 3A) and cell frequencies, absolute numbers and MFI (FIG. 3B) of Notch4 expression in lung, mediastinal lymph node (medLN) and spleen Treg and Teff cells of Foxp3^{YFPCre} mice treated with either PBS or poly I:C once daily for 6 days. FIG. 3C, Notch4 expression on lung, medLN and spleen Treg cells of Foxp3^{YFPCre} mice. FIG. 3D, M1 and M2 macrophage frequencies in cultures of Poly I:C-treated lung macrophages incubated with Treg cells from the indicated Poly I:C-treated mice. FIG. 3E, flow cytometric analysis of IL-6Rα expression in lung Notch4⁺ or Notch4⁻ Treg cells of Foxp3^{YFPCre} mice treated with poly I:C. FIG. 3F, In vitro induction of Notch4 expression in Treg cell from the lungs or spleens of Poly I:C or PBS-treated Foxp3^{YFPCre} mice. Each symbol represents one mouse (n=5-15 per group). Numbers in flow plots indicate percentages. Error bars indicate SEM. Statistical tests: Student’s T-test (b), one-way ANOVA with Dunnett’s post hoc analysis (FIG. 3D); Two-way ANOVA with Sidak’s post hoc analysis (FIGS. 3C and 3E). ***P<0.001, ****P<0.0001. Data pooled from two or three independent experiments.

FIGS. 4A-4N show impact of Treg cell-specific deletion of different Notch, Hippo and Wnt pathway components on poly I:C-induced lung injury. FIGS. 4A, 4B, Weight index (FIG. 4A) and peak weight loss (FIG. 4B) of Foxp3^{YFPCre}, Foxp3^{YFPCre}Rbpj^{Δ/Δ}, Foxp3^{YFPCre}Notch1^{Δ/Δ} Foxp3^{YFPCre}Notch2^{Δ/Δ} and Foxp3^{YFPCre}Notch3^{Δ/Δ} mice either sham-treated or treated with poly I:C, as indicated. FIG. 4C, AHR in the respective mouse groups in response to methacholine. FIGS. 4D-4F, Graphical representation of lung tissue neutrophils (FIG. 4D) and M1 (FIG. 4E) and M2 macrophages (FIG. 4F). FIGS. 4G, 4H, Flow cytometric analysis (FIG. 4G) and graphical representation (FIG. 4H) of Yap and b-Catenin in lung tissue Treg cells of Foxp3^{YFPCre} mice that were either sham-treated or treated with poly I:C. FIGS. 4I, 4J, Weight index (FIG. 4I) and peak weight loss (FIG. 4J) of Foxp3^{YFPCre}, Foxp3^{YFPCre}Yap1^{Δ/Δ}Wwtr1^{Δ/Δ}, Foxp3^{YFPCre}Ctnnb1^{Δ/Δ} and Foxp3^{YFPCre}Yap1^{Δ/Δ}Wwtr1^{Δ/Δ} Ctnnb1^{Δ/Δ} mice either sham-treated or treated with Poly I:C, as indicated. FIG. 4K, AHR in the respective mouse groups in response to methacholine. FIGS. 4L-4N, Graphical representation of lung tissue neutrophils (FIG. 4L) and M1 (FIG. 4M) and M2 (FIG. 4N) macrophages. Each symbol represents one mouse (n=5 per group). Numbers in flow plots indicate percentages. Error bars indicate SEM. Statistical tests: Two-way ANOVA with Sidak’s post hoc analysis (FIGS. 4A, 4C, 4I, 4K); One-way ANOVA with Dunnett’s post hoc analysis (FIGS. 4B, 4D, 4E, 4F, 4J, 4L, 4M, 4N);. **P<0.01, ***P<0.001, ****P<0.0001

FIGS. 5A-5H show Notch4 deficiency reprograms the lung Treg cell transcriptome of Poly I:C-treated mice. FIGS. 5A-5C, Volcano plot (FIG. 5A), heat map (FIG. 5B) and pathway analysis (FIG. 5C) of gene transcripts of lung Treg cells isolated from Foxp3^{YFPCre} and Foxp3^{YFPCre}Notch4^{Δ/Δ} mice treated with Poly I:C (n=4 and n=5, respectively). FIG. 5D, flow cytometric histograms and graphical representation of lung tissue Treg cell expression of CD25, Helios, CTLA4, ICOS, and OX40 in Foxp3^{YFPCre} and Foxp3^{YFPCre}Notch4^{Δ/Δ} mice sampled at day 7 post poly I:C treatment (n=5 for each time point). FIGS. 5E-5H, Flow cytometric analysis (FIGS. 5E, 5G) and graphical representation (FIGS. 5F, 5H) of Helios expression in Foxp3⁺Notch4⁺ and Foxp3⁺Notch4⁻ lung tissue Treg cells in Foxp3^{YFPCre} mice sampled at the indicated dates post poly I:C treatment (n=5 for each time point). Each symbol represents one mouse. Numbers in flow plots indicate percentages. Error bars indicate SEM. Statistical tests: Pairwise comparisons of differential gene expression were computed using DESeq2 (FIGS. 5A-5C); Student’s unpaired two tailed t-test (FIG. 5D) and two-way ANOVA with Sidak’s post hoc analysis (FIGS. 5F, 5H). *P<0.05, **P<0.01, ***P<0.001, ****P<0.0001

FIGS. 6A-6F show Treg cell-specific deletion of Il6ra partially protects against Poly I:C-induced lung injury. FIG. 6A, Weight index and peak weight loss of Foxp3^{YFPCre} and Foxp3YFPCreIl6ra Δ/Δ mice either sham-treated or treated with Poly I:C, as indicated. FIG. 6B, AHR in response to methacholine. FIGS. 6C, 6D, Flow cytometric analysis (FIG. 6C) and graphical representation (FIG. 6D) of Notch4 expression in lung tissue Treg cells of Foxp3^{YFPCre} and Foxp3YFPCreIl6ra Δ/Δ mice that were either sham-treated or treated with Poly I:C. FIGS. 6E-6F, Flow cytometric analysis and graphical representation of lung tissue neutrophils (FIG. 6E) and M1 (FIG. 6F) and M2 macrophages. Each symbol represents one mouse (n=5 per group). Numbers in flow plots indicate percentages. Error bars indicate SEM. Statistical tests: Two-way ANOVA with Sidak’s post hoc analysis (FIG. 6B); One-way ANOVA with Dunnett’s post hoc analysis for remaining; **P<0.01, ****P<0.0001.

FIGS. 7A-7F show validation of Notch4 agents and amphiregulin91-140 blocking peptide. FIG. 7A, Flow cytometric analysis and graphical representation of EGFR phosphorylation at tyrosine 1068 (pEGFR) in HEK293 cells treated with mouse amphiregulin in the presence increased concentrations of amphiregulin91-140 blocking peptide (bp) or a neutralizing anti-amphiregulin mAb, as indicated. FIG. 7B, Weight index and peak weight loss of FOXP3^{YFPCre} and FOXP3^{YFPCre}NOtCh4^{Δ/Δ} mice treated with Poly I:C, either alone or together with anti-amphiregulin neutralizing mAb. FIG. 7C, Frequencies of neutrophils and M1 and M2 macrophages in lung tissues. FIG. 7D, Serum tetramethylrhodamine isothiocyanate (TRITC) dextran, measured as relative fluorescent units (RFU), in Poly I:C + anti-Amphiregulin mAb-treated FOXP3^{YFPCre} or FOXP3^{YFPCre}NOtCh4^{Δ/Δ} mice at 1-hour post intra-tracheal instillation. FIG. 7E, Frequencies of neutrophils and M1 and M2 macrophages in lung tissues from FOXP3^{YFPCre} mice either sham treated or treated with Poly I:C, either alone or together with amphiregulin bp. FIG. 7F, frequencies of M1 and M2 macrophages in co-cultures of Poly I:C-treated lung macrophages incubated either alone, with amphiregulin, Treg cells from Poly I:C-treated FOXP3^{YFPCre} mice or the combination thereof. Each symbol represents one mouse (n=5-10 per group). Numbers in flow plots indicate percentages. Error bars indicate SEM. Statistical tests: One-way ANOVA with Dunnett’s post hoc analysis (FIGS. 7B-7D); two-way ANOVA with Sidak’s post hoc analysis (FIG. 7B); Student’s two tailed t-test (FIG. 7E). **P<0.01, ****P<0.0001).

FIG. 8 shows Notch4 expression on peripheral blood T cells in COVID19. Gating strategy. Peripheral blood (PB) T cells of a subject with severe COVID19 were gated with CD45 and CD3 mAbs, then CD3+CD4+ and CD3+CD4- T cells were identified. CD3+CD4+ T cells were further gated for CD25 and Foxp3 to identify Treg cells (CD4+CD25′0Foxp3-) and Tconv cells (CD4+CD25‴Foxp3- cells). The respective cell populations were analyzed for Notch4 expression, as shown.

### DETAILED DESCRIPTION

SARS-CoV-2-infected patients have CD4+ and CD8+ T cell lymphopenia that selectively affects memory cells. The basis of this lymphopenia is unknown, but may include exhaustion, cytokine damage, and fratricide by activated T cells. Epidemiologic data suggests that patients with more severe disease such as those that progress to acute respiratory distress syndrome (ARDS) have a “cytokine storm” with high levels of inflammatory cytokines, including IL-2, IP-10, MCP-1, MIP-1a TNFa, and IL-6. The possible role of this “cytokine storm” in disease pathogenesis has prompted the use of immunomodulators, especially anti-IL-6 receptor monoclonal antibody (mAb) therapy with preliminary studies reporting favorable outcome. Nevertheless, the potential for deleterious effects on antiviral defense and a higher than expected prevalence of multi-drug-resistant bacterial superinfections suggests that immunomodulatory therapy needs to be carefully balanced against its potential risks.

Relevant to the role of innate immune hyperactivation in COVID disease pathogenesis are the inventors’ previous studies on asthmatic inflammation showing how allergens and air pollution (particulate matter, PM) overcome immune tolerance mechanisms operating in the airway to license tissue inflammation. The inventors identified the JAG1-Notch4 axis as a key pathogenic mechanism activated by the allergens and PM that acts as a molecular switch to break down immune tolerance in the lung and consequently promote inflammation. Along with this mechanism, the inventors identified that Notch4 was selectively induced on lung Treg cells in an allergen and interleukin-6 (IL-6)-dependent manner, and Notch4 directed the subversion of Treg cells into Th2/Th17 effector-like T cells. Treg cell specific deletion of Notch4 inhibited airway inflammation and restored lung Treg cell regulatory functions, and this effect was recapitulated by deletion of the downstream Hippo pathway regulators Wwtrl and Yapl and the Wnt pathway regulator beta catenin-like protein 1 (Ctnnbl). Notch4-expressing lung Treg cells also fail to suppress ILC2 activation, whereas deletion of Notch4 in Treg cells restores this function. Moreover, expression of Notch4 and its down-stream Hippo and Wnt pathway effectors was increased on circulating Treg cells of asthmatics as a function of disease severity, in association with reduced Treg cell-mediated suppression.

Importantly, the inventors have identified that Notch4 expression is selectively upregulated on circulating Treg cells of COVID19 subjects as a function of disease severity, and Notch4 expression precipitously declines following patient recovery, thus implicating this mechanism in disease pathogenesis in COVID19 subjects. Data presented herein suggest that Notch4 is a biomarker for COVID-19 disease severity, as well as a therapeutic target whose targeting would render Tregs functional and thus suppress the cytokine storm in the lungs of COVID-19 patients.

Accordingly, provided herein are methods for treating or ameliorating a symptom of a coronavirus infectious disease (e.g., COVID-19) by administering to a subject having a coronavirus infection disease a Notch4 modulating agent (e.g., an agent that inhibits Notch4). Additionally, provided herein are methods for preventing a coronavirus infectious disease (e.g., COVID-19) by administering to a subject at risk of developing a coronavirus infection disease a Notch4 modulating agent (e.g., an agent that inhibits Notch4).

Compositions comprising a Notch4 modulating agent (e.g., an agent that inhibits Notch4) for use in treating or preventing a coronavirus infectious disease are further provided.

### Notch4

The Notch signaling pathway is an evolutionarily conserved intercellular signaling pathway that regulates interactions between physically adjacent cells. Notch signaling regulates multiple cell fate decisions; each Notch family member plays a role in a variety of developmental processes. In mammals, the Notch family is composed of four Notch receptors (Notch1-Notch4) and five ligands [Delta-like ligand 1 (DLL1), DLL3, DLL4, Jagged(Jag)1 and Jag2]. Upon binding to Jagged or Delta-like ligands on an adjacent cell, two sequential proteolytic events release the intracellular domain of Notch (NICD) allowing its translocation to the nucleus. There the NICD converts the DNA binding factor RBP-J from a transcriptional repressor to a transcriptional activator through MAML1-MAML3 binding.

The NOTCH protein is cleaved in the trans-Golgi network, and then presented on the cell surface as a heterodimer. The protein functions as a receptor for membrane bound ligands, and may play a role in vascular, renal, and hepatic development.

SEQ ID NO: 1 contains a nucleic acid sequence that encodes Notch 4.

a tgcagccccc ttcactgctg ctgctgctgc
tgctgctgct gctgctatgt gtctcagtgg tcagacccag agggctgctg
tgtgggagtt tcccagaacc ctgtgccaat ggaggcacct gcctgagcct
gtctctggga caagggacct gccagtgtgc ccctggcttc ctgggtgaga
cgtgccagtt tcctgacccc tgccagaacg cccagctctg ccaaaatgga
ggcagctgcc aagccctgct tcccgctccc ctagggctcc ccagctctcc
ctctccattg acacccagct tcttgtgcac ttgcctccct ggcttcactg
gtgagagatg ccaggccaag cttgaagacc cttgtcctcc ctccttctgt
tccaaaaggg gccgctgcca catccaggcc tcgggccgcc cacagtgctc
ctgcatgcct ggatggacag gtgagcagtg ccagcttcgg gacttctgtt
cagccaaccc atgtgttaat ggaggggtgt gtctggccac atacccccag
atccagtgcc actgcccacc gggcttcgag ggccatgcct gtgaacgtga
tgtcaacgag tgcttccagg acccaggacc ctgccccaaa ggcacctcct
gccataacac cctgggctcc ttccagtgcc tctgccctgt ggggcaggag
ggtccacgtt gtgagctgcg ggcaggaccc tgccctccta ggggctgttc
gaatgggggc acctgccagc tgatgccaga gaaagactcc acctttcacc
tctgcctctg tcccccaggt ttcataggcc cagactgtga ggtgaatcca
gacaactgtg tcagccacca gtgtcagaat gggggcactt gccaggatgg
gctggacacc tacacctgcc tctgcccaga aacctggaca ggctgggact
gctccgaaga tgtggatgag tgtgagaccc agggtccccc tcactgcaga
aacgggggca cctgccagaa ctctgctggt agctttcact gcgtgtgtgt
gagtggctgg ggcggcacaa gctgtgagga gaacctggat gactgtattg
ctgccacctg tgccccggga tccacctgca ttgaccgggt gggctctttc
tcctgcctct gcccacctgg acgcacagga ctcctgtgcc acttggaaga
catgtgtctg agccagccgt gccatgggga tgcccaatgc agcaccaacc
ccctcacagg ctccacactc tgcctgtgtc agcctggcta ttcggggccc
acctgccacc aggacctgga cgagtgtctg atggcccagc aaggcccaag
tccctgtgaa catggcggtt cctgcctcaa cactcctggc tccttcaact
gcctctgtcc acctggctac acaggctccc gttgtgaggc tgatcacaat
gagtgcctct cccagccctg ccacccagga agcacctgtc tggacctact
tgccaccttc cactgcctct gcccgccagg cttagaaggg cagctctgtg
aggtggagac caacgagtgt gcctcagctc cctgcctgaa ccacgcggat
tgccatgacc tgctcaacgg cttccagtgc atctgcctgc ctggattctc
cggcacccga tgtgaggagg atatcgatga gtgcagaagc tctccctgtg
ccaatggtgg gcagtgccag gaccagcctg gagccttcca ctgcaagtgt
ctcccaggct ttgaagggcc acgctgtcaa acagaggtgg atgagtgcct
gagtgaccca tgtcccgttg gagccagctg ccttgatctt ccaggagcct
tcttttgcct ctgcccctct ggtttcacag gccagctctg tgaggttccc
ctgtgtgctc ccaacctgtg ccagcccaag cagatatgta aggaccagaa
agacaaggcc aactgcctct gtcctgatgg aagccctggc tgtgccccac
ctgaggacaa ctgcacctgc caccacgggc actgccagag atcctcatgt
gtgtgtgacg tgggttggac ggggccagag tgtgaggcag agctaggggg
ctgcatctct gcaccctgtg cccatggggg gacctgctac ccccagccct
ctggctacaa ctgcacctgc cctacaggct acacaggacc cacctgtagt
gaggagatga cagcttgtca ctcagggcca tgtctcaatg gcggctcctg
caaccctagc cctggaggct actactgcac ctgccctcca agccacacag
ggccccagtg ccaaaccagc actgactact gtgtgtctgc cccgtgcttc
aatgggggta cctgtgtgaa caggcctggc accttctcct gcctctgtgc
catgggcttc cagggcccgc gctgtgaggg aaagctccgc cccagctgtg
cagacagccc ctgtaggaat agggcaacct gccaggacag ccctcagggt
ccccgctgcc tctgccccac tggctacacc ggaggcagct gccagactct
gatggactta tgtgcccaga agccctgccc acgcaattcc cactgcctcc
agactgggcc ctccttccac tgcttgtgcc tccagggatg gaccgggcct
ctctgcaacc ttccactgtc ctcctgccag aaggctgcac tgagccaagg
catagacgtc tcttcccttt gccacaatgg aggcctctgt gtcgacagcg
gcccctccta tttctgccac tgcccccctg gattccaagg cagcctgtgc
caggatcacg tgaacccatg tgagtccagg ccttgccaga acggggccac
ctgcatggcc cagcccagtg ggtatctctg ccagtgtgcc ccaggctacg
atggacagaa ctgctcaaag gaactcgatg cttgtcagtc ccaaccctgt
cacaaccatg gaacctgtac tcccaaacct ggaggattcc actgtgcctg
ccctccaggc tttgtggggc tacgctgtga gggagacgtg gacgagtgtc
tggaccagcc ctgccacccc acaggcactg cagcctgcca ctctctggcc
aatgccttct actgccagtg tctgcctgga cacacaggcc agtggtgtga
ggtggagata gacccctgcc acagccaacc ctgctttcat ggagggacct
gtgaggccac agcaggatca cccctgggtt tcatctgcca ctgccccaag
ggttttgaag gccccacctg cagccacagg gccccttcct gcggcttcca
tcactgccac cacggaggcc tgtgtctgcc ctcccctaag ccaggcttcc
caccacgctg tgcctgcctc agtggctatg ggggtcctga ctgcctgacc
ccaccagctc ctaaaggctg tggccctccc tccccatgcc tatacaatgg
cagctgctca gagaccacgg gcttgggggg cccaggcttt cgatgctcct
gccctcacag ctctccaggg ccccggtgtc agaaacccgg agccaagggg
tgtgagggca gaagtggaga tggggcctgc gatgctggct gcagtggccc
gggaggaaac tgggatggag gggactgctc tctgggagtc ccagacccct
ggaagggctg cccctcccac tctcggtgct ggcttctctt ccgggacggg
cagtgccacc cacagtgtga ctctgaagag tgtctgtttg atggctacga
ctgtgagacc cctccagcct gcactccagc ctatgaccag tactgccatg
atcacttcca caacgggcac tgtgagaaag gctgcaacac tgcagagtgt
ggctgggatg gaggtgactg caggcctgaa gatggggacc cagagtgggg
gccctccctg gccctgctgg tggtactgag ccccccagcc ctagaccagc
agctgtttgc cctggcccgg gtgctgtccc tgactctgag ggtaggactc
tgggtaagga aggatcgtga tggcagggac atggtgtacc cctatcctgg
ggcccgggct gaagaaaagc taggaggaac tcgggacccc acctatcagg
agagagcagc ccctcaaacg cagcccctgg gcaaggagac cgactccctc
agtgctgggt ttgtggtggt catgggtgtg gatttgtccc gctgtggccc
tgaccacccg gcatcccgct gtccctggga ccctgggctt ctactccgct
tccttgctgc gatggctgca gtgggagccc tggagcccct gctgcctgga
ccactgctgg ctgtccaccc tcatgcaggg accgcacccc ctgccaacca
gcttccctgg cctgtgctgt gctccccagt ggccggggtg attctcctgg
ccctaggggc tcttctcgtc ctccagctca tccggcgtcg acgccgagag
catggagctc tctggctgcc ccctggtttc actcgacggc ctcggactca
gtcagctccc caccgacgcc ggcccccact aggcgaggac agcattggtc
tcaaggcact gaagccaaag gcagaagttg atgaggatgg agttgtgatg
tgctcaggcc ctgaggaggg agaggaggtg ggccaggctg aagaaacagg
cccaccctcc acgtgccagc tctggtctct gagtggtggc tgtggggcgc
tccctcaggc agccatgcta actcctcccc aggaatctga gatggaagcc
cctgacctgg acacccgtgg acctgatggg gtgacacccc tgatgtcagc
agtttgctgt ggggaagtac agtccgggac cttccaaggg gcatggttgg
gatgtcctga gccctgggaa cctctgctgg atggaggggc ctgtccccag
gctcacaccg tgggcactgg ggagaccccc ctgcacctgg ctgcccgatt
ctcccggcca accgctgccc gccgcctcct tgaggctgga gccaacccca
accagccaga ccgggcaggg cgcacacccc ttcatgctgc tgtggctgct
gatgctcggg aggtctgcca gcttctgctc cgtagcagac aaactgcagt
ggacgctcgc acagaggacg ggaccacacc cttgatgctg gctgccaggc
tggcggtgga agacctggtt gaagaactga ttgcagccca agcagacgtg
ggggccagag ataaatgggg gaaaactgcg ctgcactggg ctgctgccgt
gaacaacgcc cgagccgccc gctcgcttct ccaggccgga gccgataaag
atgcccagga caacagggag cagacgccgc tattcctggc ggcgcgggaa
ggagcggtgg aagtagccca gctactgctg gggctggggg cagcccgaga
gctgcgggac caggctgggc tagcgccggc ggacgtcgct caccaacgta
accactggga tctgctgacg ctgctggaag gggctgggcc accagaggcc
cgtcacaaag ccacgccggg ccgcgaggct gggcccttcc cgcgcgcacg
gacggtgtca gtaagcgtgc ccccgcatgg gggcggggct ctgccgcgct
gccggacgct gtcagccgga gcaggccctc gtgggggcgg agcttgtctg
caggctcgga cttggtccgt agacttggct gcgcgggggg gcggggccta
ttctcattgc cggagcctct cgggagtagg agcaggagga ggcccgaccc
ctcgcggccg taggttttct gcaggcatgc gcgggcctcg gcccaaccct
gcgataatgc gaggaagata cggagtggct gccgggcgcg gaggcagggt
ctcaacggat gactggccct gtgattgggt ggccctggga gcttgcggtt
ctgcctccaa cattccgatc ccgcctcctt g (SEQ ID NO: 1)

### Coronavirus Infectious Disease

Coronaviruses belong to the subfamily Coronavirinae in the family Coronaviridae and are named for the crown-like spikes on their surface. There are four main sub-groupings of coronaviruses, known as alpha, beta, gamma, and delta. Coronaviruses viruses typically affect the respiratory tracts of birds and mammals, including humans. The most recent data suggest that there are 7 coronaviruses that are capable of infecting humans.

Globally, people are commonly infected with human coronaviruses 229E, NL63, OC43, and HKU1, resulting in the common cold. Infection by these coronaviruses most often occur during the winter months and early spring. At times, a coronavirus that previously infected an animal will infect a human, resulting in a new human coronavirus. These new viruses are more rare, but can result in a more severe infection. Three recent examples of this are SARS-CoV-2 (the novel coronavirus that causes coronavirus disease 2019 (COVID-19), SARS-CoV (the beta coronavirus that causes severe acute respiratory syndrome, or SARS), and MERS-CoV (the beta coronavirus that causes Middle East Respiratory Syndrome, or MERS).

Symptoms associated with a typical coronavirus infectious disease (e.g., the common cold) include runny nose, headache, cough, fever, and sore throat. There is currently no cure for the common cold; treatments typically include self-care and over-the-counter medications to manage symptoms.

Symptoms associated with an infection by SARS-CoV-2, resulting in COVID-19, include fever, chills, persistent dry cough, shortness of breath, sore throat, headache, loss of taste or smell, and gastrointestinal distress. The development of a serious illness and/or a poor outcome from COVID-19 is most commonly observed in subjects over the age of 65, having chronic lung disease, serious heart conditions, severe obesity, a compromised immune system, or diabetes, and living in a nursing home or care facility. COVID-19 is rarely observed in subjects under the age of 18.

Current treatments for COVID-19 are designed to treat individual symptoms, for example, a corticosteroid inhaler is prescribed to a subject having difficulty breathing, or a ventilator can be used for a subject having a serious illness related to COVID-19. Plasma from a subject that has been previously diagnosed with having COVID-19, and has recovered from COVD-19, has been administered as a COVID-19 therapeutic.

Subjects diagnosed with having a coronavirus infectious disease (e.g., COVID-19) are at risk of further developing acute respiratory distress syndrome (ARDS). ARDS is a life threatening syndrome that occurs when fluid builds up in the alveoli in your lungs. The fluid keeps your lungs from filling with enough air, resulting in a markedly reduced level of oxygen reaching your bloodstream, depriving your organs of the oxygen. ARDS typically occurs in people who are already critically ill or who have significant injuries. Symptoms of ARDS usually develops within a few hours to a few days after the precipitating injury or infection. The risk of death associated with ARDS increases with age (those 60 years of age and older are at a greater risk of death) and severity of illness. Of the people who do survive ARDS, some recover completely while others experience lasting damage to their lungs.

The most common underlying causes and risk factors of ARDS include sepsis (i.e., a serious and widespread infection of the bloodstream); inhalation of harmful substances or aspirating vomit or near-drowning episodes; severe pneumonia, e.g., pneumonia that affects all five lobes of the lungs; head, chest or other major injury, e.g., that directly damage the lungs or the portion of the brain that controls breathing; pancreatitis; massive blood transfusions; and burns. ARDS can be diagnosed by a skilled clinician by determining if a subject exhibit at least one symptom of ARDS, e.g., shortness of breath, labored and unusually rapid breathing, low blood pressure, and confusion and extreme tiredness.

Subjects with ARDS are at a greater risk of developing blood clots, collapsed lung, secondary infections, and pulmonary fibrosis.

### Treating or Preventing Coronavirus Infectious Disease

One aspect of the invention provided herein is a method of treating a coronavirus infectious disease by administering to a subject having coronavirus infectious disease a Notch4 modulating agent (e.g., an agent that inhibits Notch4). In one embodiment of any aspect, the method further comprises the step of, prior to administering, diagnosing the subject as having coronavirus infectious disease. In one embodiment of any aspect, the method further comprises the step of, prior to administering, receiving the results of an assay that diagnoses the subject as having coronavirus infectious disease.

A skilled clinician can diagnose a subject as having a coronavirus infectious disease, e.g., by determining if the subject presents with at least one (1) symptom associated with coronavirus infectious disease, e.g., runny nose, headache, cough, fever, and sore throat. Diagnostic tests useful in identifying a subject as having coronavirus infectious disease are known in the art and include, but are not limited to, RNA sequencing of a sample to assess for the presence of a coronavirus in the sample.

Another aspect provides a method for treating COVID-19 comprising administering to a subject having COVID-19 an effective amount of a Notch4 modulating agent (e.g., an agent that inhibits Notch4). In one embodiment of any aspect, the method further comprises the step of, prior to administering, diagnosing the subject as having COVID-19. In one embodiment of any aspect, the method further comprises the step of, prior to administering, receiving the results of an assay that diagnoses the subject as having COVID-19.

A skilled clinician can diagnose a subject as having COVID-19, e.g., by determining if the subject presents with at least one (1) symptom associated with COVID-19, e.g., fever, dry cough, loss of taste or small, fatigue, and pneumonia. Diagnostic tests useful in identifying a subject as having coronavirus infectious disease include, but is not limited to, a nasopharyngeal swab.

Another aspect of the invention herein is a method of preventing a coronavirus infectious disease, comprising administering to a subject at risk of developing a coronavirus infectious disease a Notch4 modulating agent (e.g., an agent that inhibits Notch4). In one embodiment, the method further comprises, prior to administering, identifying a subject at risk of developing coronavirus infectious disease prior to administering the agent. In one embodiment, the method further comprises, prior to administering, receiving the results of an assay that identifies a subject as being at risk of developing coronavirus infectious disease prior to administering the agent.

As used herein a subject “at risk of developing coronavirus infectious disease” refers to a subject who has been in contact, or potentially in contact, with a subject having a coronavirus infectious disease. Transmission of the coronavirus infectious disease causing virus is often airborne, moving in liquid droplets, or transmitted from a surface, thus close contact with a coronavirus infectious disease-positive subject increases the likelihood of developing the disease. A skilled person can determine if a person is at risk of developing a coronavirus infectious disease by determining if the subject has been around a coronavirus infectious disease-positive person. If a subject has been in contact with a person that has received the results of an assay that diagnoses the person as having a coronavirus infectious disease, this is sufficient to diagnose the subject as being at risk of developing a coronavirus infectious disease.

Another aspect of the invention herein is a method of preventing COVID-19, comprising administering to a subject at risk of developing COVID-19 a Notch4 modulating agent (e.g., an agent that inhibits Notch4). In one embodiment, the method further comprises, prior to administering, identifying a subject at risk of developing COVID-19. In one embodiment, the method further comprises, prior to administering, receiving the results of an assay that identifies a subject as being at risk of developing COVID-19.

As used herein a subject “at risk of developing coronavirus infectious disease” refers to a subject who has been in contact, or potentially in contact, with a subject having a coronavirus infectious disease. Transmission of the COVID-19 causing virus is airborne, moving in liquid droplets, thus close contact with a COVID-19 positive subject increases the likelihood of developing the disease. A skilled person can determine if a person is at risk of developing COVID-19 by determining if the subject has been around a COVID-19-positive person. If a subject has been in contact with a person that has received the results of an assay that diagnoses the person as having a COVID-19, this is sufficient to diagnose the subject as being at risk of developing COVID-19.

For example, if a person that the subject was in close contact with is diagnosed with COVID-19 via a swab assay, this swab assay result would be sufficient to determine that the subject is at risk of developing COVID-19. In a further example, if a person that the subject was in close contact with told the subject that they were COVID-19-positive, this verbal confirmation would be sufficient to determine that the subject is at risk of developing COVID-19. If a subject is contacted by a public health official and told that they were in close contact with a COVID-19-positive, this verbal confirmation would be sufficient to determine that the subject is at risk of developing COVID-19.

Risk factors for COVID-19 described herein above can also be used to determine if a subject is at risk for developing COVID-19.

In one embodiment, the methods described herein further comprise administering at least one additional therapeutic. In one embodiment, the at least one additional therapeutic is an anti-viral therapeutic. Anti-viral therapeutics are known in the art, and are further provided herein below.

### Agents

As used herein, the term “Notch4 modulating agent” refers to any agent that is capable of being used to modulate expression, activity, and/or function of Notch4. Notch4 modulating agents include but are not limited to agents that inhibit Notch4. In some embodiments, the Notch4 modulating agent is capable of binding to Notch4. In some embodiments, the Notch4 modulating agent is capable of binding directly to Notch4. In one aspect, a Notch4 modulating agent (e.g., an agent that inhibits Notch4) is administered to a subject having, or at risk of having a coronavirus infectious disease, e.g., COVID-19. In one embodiment, the Notch4 modulating agent (e.g., agent that inhibits Notch4) is a small molecule, an antibody or antibody fragment, a peptide, an antisense oligonucleotide, a genome editing system, or an RNAi.

An agent is considered effective for modulating (or inhibiting) Notch4 if, for example, upon administration, it modulates (or inhibits) the presence, amount, activity and/or level of Notch4 in the cell.

In one embodiment, upon administration, the agent modulates or inhibits the presence, amount, activity and/or level of Notch4 in the cell by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to an appropriate control or reference level. In some embodiments, an appropriate control would be the presence, amount, activity and/or level of Notch4 in a substantially identical cell that is not administered an agent described herein.

In one embodiment, modulating or inhibiting Notch4 comprises inhibiting the differentiation of a Notch4-expressing Treg cell into a disease-promoting Th cell.

An agent can modulate or inhibit e.g., the transcription, or the translation of Notch4 in the cell. An agent can modulate or inhibit the activity or alter the activity (e.g., such that the activity no longer occurs, or occurs at a reduced rate) of Notch4 in the cell (e.g., Notch4′s expression).

In one embodiment of any aspect, Notch4 is modulated or inhibited on T regulatory cells. In one embodiment, a Notch4 modulating agent (e.g., an agent that inhibits Notch4) promotes programmed cell death, e.g., kill, the cell that expresses Notch4, for example, a T reg cell. To determine whether an agent is effective at modulating or inhibiting Notch4, mRNA and protein levels of a given target (e.g., Notch4) can be assessed using RT-PCR and western-blotting, respectively. Biological assays that detect the activity of Notch4 (e.g., Notch reporters that measure the binding of the Notch receptor and ligand) can be used to assess if programmed cell death has occurred. Alternatively, immunofluorescence detection using antibodies specific to Notch4 in combination with cell death markers (e.g., Caspase) can be used to determine if cell death has occurred following administration of an agent.

In one embodiment, an agent that modulates or inhibits the level and/or activity of Notch4 by at least 10%, by at least 20%, by at least 30%, by at least 40%, by at least 50%, by at least 60%, by at least 70%, by at least 80%, by at least 90%, by at least 100% or more as compared to an appropriate control or reference level. In some embodiments, an “appropriate control” refers to the level and/or activity of Notch4 prior to administration of the agent, or the level and/or activity of Notch4 in a population of cells that was not in contact with the agent.

The agent may function directly in the form in which it is administered. Alternatively, the agent can be modified or utilized intracellularly to produce something which modulates or inhibits Notch4, such as introduction of a nucleic acid sequence into the cell and its transcription resulting in the production of the nucleic acid and/or protein modulator or inhibitor of Notch4. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be identified from a library of diverse compounds.

In various embodiments, the agent is a small molecule that modulates or inhibits Notch4. Methods for screening small molecules are known in the art and can be used to identify a small molecule that is efficient at, for example, inducing cell death of pathogenic CD4 cells, given the desired target (e.g., Notch4).

In various embodiments, the Notch4 modulating agent (e.g., agent that inhibits Notch4) is an antibody or antigen-binding fragment thereof, or an antibody reagent that is specific for Notch4. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

In one embodiment, the Notch4 modulating agent (e.g., agent that inhibits Notch4) is a humanized, monoclonal antibody or antigen-binding fragment thereof, or an antibody reagent. As used herein, “humanized” refers to antibodies from non-human species (e.g., mouse, rat, sheep, etc.) whose protein sequence has been modified such that it increases the similarities to antibody variants produce naturally in humans. In one embodiment, the humanized antibody is a humanized monoclonal antibody. In one embodiment, the humanized antibody is a humanized polyclonal antibody. In one embodiment, the humanized antibody is for therapeutic use.

In one embodiment, the antibody or antibody reagent binds to an amino acid sequence that corresponds to the amino acid sequence encoding Notch4 (SEQ ID NO: 2).

MQPPSLLLLLLLLLLLCVSVVRPRGLLCGSFPEPCANGGTCLSLSLGQGT
CQCAPGFLGETCQFPDPCQNAQLCQNGGSCQALLPAPLGLPSSPSPLTPS
FLCTCLPGFTGERCQAKLEDPCPPSFCSKRGRCHIQASGRPQCSCMPGWT
GEQCQLRDFCSANPCVNGGVCLATYPQIQCHCPPGFEGHACERDVNECFQ
DPGPCPKGTSCHNTLGSFQCLCPVGQEGPRCELRAGPCPPRGCSNGGTCQ
LMPEKDSTFHLCLCPPGFIGPDCEVNPDNCVSHQCQNGGTCQDGLDTYTC
LCPETWTGWDCSEDVDECETQGPPHCRNGGTCQNSAGSFHCVCVSGWGGT
SCEENLDDCIAATCAPGSTCIDRVGSFSCLCPPGRTGLLCHLEDMCLSQP
CHGDAQCSTNPLTGSTLCLCQPGYSGPTCHQDLDECLMAQQGPSPCEHGG
SCLNTPGSFNCLCPPGYTGSRCEADHNECLSQPCHPGSTCLDLLATFHCL
CPPGLEGQLCEVETNECASAPCLNHADCHDLLNGFQCICLPGFSGTRCEE
DIDECRSSPCANGGQCQDQPGAFHCKCLPGFEGPRCQTEVDECLSDPCPV
GASCLDLPGAFFCLCPSGFTGQLCEVPLCAPNLCQPKQICKDQKDKANCL
CPDGSPGCAPPEDNCTCHHGHCQRSSCVCDVGWTGPECEAELGGCISAPC
AHGGTCYPQPSGYNCTCPTGYTGPTCSEEMTACHSGPCLNGGSCNPSPGG
YYCTCPPSHTGPQCQTSTDYCVSAPCFNGGTCVNRPGTFSCLCAMGFQGP
RCEGKLRPSCADSPCRNRATCQDSPQGPRCLCPTGYTGGSCQTLMDLCAQ
KPCPRNSHCLQTGPSFHCLCLQGWTGPLCNLPLSSCQKAALSQGIDVSSL
CHNGGLCVDSGPSYFCHCPPGFQGSLCQDHVNPCESRPCQNGATCMAQPS
GYLCQCAPGYDGQNCSKELDACQSQPCHNHGTCTPKPGGFHCACPPGFVG
LRCEGDVDECLDQPCHPTGTAACHSLANAFYCQCLPGHTGQWCEVEIDPC
HSQPCFHGGTCEATAGSPLGFICHCPKGFEGPTCSHRAPSCGFHHCHHGG
LCLPSPKPGFPPRCACLSGYGGPDCLTPPAPKGCGPPSPCLYNGSCSETT
GLGGPGFRCSCPHSSPGPRCQKPGAKGCEGRSGDGACDAGCSGPGGNWDG
GDCSLGVPDPWKGCPSHSRCWLLFRDGQCHPQCDSEECLFDGYDCETPPA
CTPAYDQYCHDHFHNGHCEKGCNTAECGWDGGDCRPEDGDPEWGPSLALL
VVLSPPALDQQLFALARVLSLTLRVGLWVRKDRDGRDMVYPYPGARAEEK
LGGTRDPTYQERAAPQTQPLGKETDSLSAGFVWMGVDLSRCGPDHPASRC
PWDPGLLLRFLAAMAAVGALEPLLPGPLLAVHPHAGTAPPANQLPWPVLC
SPVAGVILLALGALLVLQLIRRRRREHGALWLPPGFTRRPRTQSAPHRRR
PPLGEDSIGLKALKPKAEVDEDGWMCSGPEEGEEVGQAEETGPPSTCQLW
SLSGGCGALPQAAMLTPPQESEMEAPDLDTRGPDGVTPLMSAVCCGEVQS
GTFQGAWLGCPEPWEPLLDGGACPQAHTVGTGETPLHLAARFSRPTAARR
LLEAGANPNQPDRAGRTPLHAAVAADAREVCQLLLRSRQTAVDARTEDGT
TPLMLAARLAVEDLVEELIAAQADVGARDKWGKTALHWAAAVNNARAARS
LLQAGADKDAQDNREQTPLFLAAREGAVEVAQLLLGLGAARELRDQAGLA
PADVAHQRNHWDLLTLLEGAGPPEARHKATPGREAGPFPRARTVSVSVPP
HGGGALPRCRTLSAGAGPRGGGACLQARTWSVDLAARGGGAYSHCRSLSG
VGAGGGPTPRGRRFSAGMRGPRPNPAIMRGRYGVAAGRGGRVSTDDWPCD
WVALGACGSASNIPIPPPCLTPSPERGSPQLDCGPPALQEMPINQGGEGK
K (SEQ ID NO: 2)

In another embodiment, the anti-Notch4 antibody or antibody reagent binds to an amino acid sequence that comprises the sequence of SEQ ID NO: 2; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 2. In one embodiment, the anti-Notch4 antibody or antibody reagent binds to an amino acid sequence that comprises the entire sequence of SEQ ID NO: 2. In another embodiment, the antibody or antibody reagent binds to an amino acid sequence that comprises a fragment of the sequence of SEQ ID NO: 2, wherein the fragment is sufficient to bind its target, e.g., Notch4, and modulates or inhibits the differentiation of a Notch4-expressing Treg cell into a disease-promoting Th cell.

In one embodiment, the Notch4 modulating agent (e.g., agent that inhibits Notch4) is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under cellular conditions to a gene, e.g., Notch4. Thus, oligonucleotides are chosen that are sufficiently complementary to the target, i.e., that hybridize sufficiently well and with sufficient specificity in the context of the cellular environment, to give the desired effect. For example, an antisense oligonucleotide that modulates inhibits Notch4 may comprise at least 5, at least 10, at least 15, at least 20, at least 25, at least 30, or more bases complementary to a portion of the coding sequence of the human Notch4 gene (e.g., SEQ ID NO: 1). Examples of Notch4 modulating antisense oligonucleotides are known in the art, e.g., as described in US 2020-0171071 A1, the contents of which are hereby incorporated by reference in its entirty.

In one embodiment, Notch4 is are depleted from the cell’s genome using any genome editing system including, but not limited to, zinc finger nucleases, TALENS, meganucleases, and CRISPR/Cas systems. In one embodiment, the genomic editing system used to incorporate the nucleic acid encoding one or more guide RNAs into the cell’s genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference. The CRISPR/Cas system is originally an RNA-mediated bacterial immune system that provides a form of acquired immunity against viruses and plasmids; it comprises three components: a Cas (CRISPR associated protein) endonuclease (such as Streptococcus pyogenes Cas9 or Francisella novicida Cas12a), a crRNA (CRISPR RNA), and a tracrRNA (transactivating crRNA). Clustered regularly interspaced short palindromic repeats (CRISPR) are short repetitions of bacterial DNA followed by short repetitions of spacer DNA from viruses or plasmids. The Cas9 endonuclease contains two nuclease domains and is programmed by a crRNA and tracrRNA hybrid to cleave the target sequence. In some embodiments, the Cas9 endonuclease is programmed by a crRNA and tracrRNA hybrid to cleave, e.g., a Notch4 sequence. In other embodiments, the Cas9 endonuclease is programmed by a single-guide RNA (sgRNA), which contains both a crRNA and tracrRNA sequence. In some cases, the guide RNAs (gRNAs) are selected to generate a functional gene deletion, in other cases the gRNAs are selected to recruit a catalytically inactive Cas molecule to inhibit transcription of the target loci (CRISPR interference; CRISPRi) or activate transcription of human target loci (CRISPR activation; CRISPRa).

There are two main considerations in the selection of the 20-nt guide sequence for gene targeting: 1) the target sequence should precede the protospacer adjacent motif (PAM) sequence specific for the Cas nucleus used (5′- GG PAM for S. pyogenes Cas9), and 2) guide sequences should be chosen to minimize off-target activity. Guide RNA sequences can be readily generated for a given target sequence using prediction software, for example, CRISPRdirect (available on the world wide web at http://crispr.dbels.jp/), see Natio, et al. Bioinformatics. (2015) Apr 1; 31(7): 1120-1123; ATUM gRNA Design Tool (available on the world wide web at www.atum.bio:ecommerce/cas9/input); an CRISPR-ERA (available on the world wide web at http://crispr-era.stanford.eduu/indexjsp), see Liu, et al. Bioinformatics, (2015) Nov 15; 31(22): 3676-3678. All references cited herein are incorporated herein by reference in their entireties. Non-limiting examples of publicly available gRNA design software include; sgRNA Scorer 1.0, Quilt Universal guide RNA designer, Cas-OFFinder & Cas-Designer, CRISPR-ERA, CRISPR/Cas9 target online predictor, Off-Spotter - for designing gRNAs, CRISPR MultiTargeter, ZiFiT Targeter, CRISPRdirect, CRISPR design from crispr.mit.edu/, E-CRISP etc.

A CRISPR/Cas system can be delivered using a plasmid, vector, or a ribonucleoprotein complex. Ribonucleoprotein complexes comprising a Cas protein can further comprise a nucleic acid sequence encoding crRNA and tracrRNA. When a nucleic acid encoding one or more sgRNAs and a nucleic acid encoding an RNA-guided endonuclease each need to be administered, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

In one embodiment, the agent modulates or inhibits Notch4 by RNA modulation or inhibition. Modulators or inhibitors of the expression of a given gene can be, e.g., modulatory or inhibitory nucleic acids. In some embodiments of any of the aspects, the inhibitory nucleic acid is an inhibitory RNA (iRNA). The RNAi can be single stranded or double stranded.

The iRNA can be siRNA, shRNA, endogenous microRNA (miRNA), or artificial miRNA. In one embodiment, an iRNA as described herein effects inhibition of the expression and/or activity of a target, e.g. Notch4. In some embodiments of any of the aspects, the agent is siRNA that inhibits Notch4. In some embodiments of any of the aspects, the agent is shRNA that inhibits Notch4.

One skilled in the art would be able to design siRNA, shRNA, or miRNA to target Notch4, e.g., using publically available design tools. siRNA, shRNA, or miRNA is commonly made using algortihms, such as RNAi Design (available, e.g., on the world wide web at rnaidesigner.thermofisher.com/maiexpress/ and on the world wide web at biotools.idtdna.com/site/order/designtool/index/DSIRNA_CUSTOM); Dharmacon (Layfayette, CO) (available, e.g., on the world wide web at https://www.thermofisher.com/order/custom-genomic-products/tools/sirna/); or Sigma Aldrich (St. Louis, MO) (available, e.g., on the world wide web at https://www.sigmaaldrich.com/life-science/custom-oligos/sirna-oligos/sirna-design-service.html).

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The RNA of an iRNA can be chemically modified to enhance stability or other beneficial characteristics. The nucleic acids featured in the invention may be synthesized and/or modified by methods well established in the art, such as those described in “Current protocols in nucleic acid chemistry,” Beaucage, S.L. et al. (Edrs.), John Wiley & Sons, Inc., New York, NY, USA, which is hereby incorporated herein by reference.

In one embodiment, the agent is miRNA that modulates or inhibits Notch4. microRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector.

The agent may result in gene silencing of the target gene (e.g., Notch4), such as with an RNAi molecule (e.g. siRNA or miRNA). This entails a decrease in the mRNA level in a cell for a target by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more of the mRNA level found in the cell without the presence of the agent. In one preferred embodiment, the mRNA levels are decreased by at least about 70%, about 80%, about 90%, about 95%, about 99%, about 100%. One skilled in the art will be able to readily assess whether the siRNA, shRNA, or miRNA effective target e.g., Notch4, for its downregulation, for example by transfecting the siRNA, shRNA, or miRNA into cells and detecting the levels of a gene (e.g., Notch4) found within the cell via western-blotting.

The agent may be contained in and thus further include a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., a Notch4 modulating agent, e.g., Notch4 inhibitor) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

In one embodiment, the methods require or involve the administration of an agent that inhibits or reduces a target downstream of Notch4, e.g., downstream Hippo pathway regulators Wwtrl and Yapl and Wnt pathway Ctnnbl.

Further provided herein is a composition for the treatment or prevention of a coronavirus infectious disease comprising any of the agents that modulates or inhibits Notch4 described herein and a pharmaceutically acceptable carrier.

Further provided herein is a composition for the treatment or prevention of a COVID-19 comprising any of the agents that modulates or inhibits Notch4 described herein and a pharmaceutically acceptable carrier.

In certain embodiment, compositions described herein are formulated for inhaled or aerosol administration for local delivery of the composition.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, an agent as described herein. Each carrier must be “acceptable” in the sense of being compatible with the other ingredients of the formulation and not injurious to the patient. The terms “excipient,” “carrier,” “pharmaceutically acceptable carrier” or the like are used interchangeably herein.

### Exemplary Agents That Modulate or Inhibit Notch4

Small molecule modulators or inhibitors of Notch4 are known in the art and may be useful in the present invention. For example, DAPT (Item no. D5942 from Sigma Aldrich). DAPT, also known as GSI-IX; LY-374973; N-[N-(3,5-Difluorophenacetyl)-L-alanyl]-S-phenylglycine t-butyl ester; has a chemical structure of

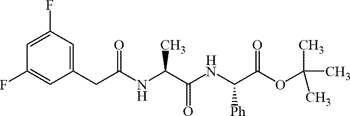 [SMILES: C[C@H](NC(=O)Cc1cc(F)cc(F)c1)C(=O)N[C@H](C(=O)OC(C)(C)C)c1ccccc1]

An additional exemplary small molecule inhibitor of Notch4 includes L-685,458 (Item no. L1790 from Sigma Aldrich). L-685,458, also known as (5S)-(t-Butoxycarbonylamino)-6-phenyl-(4R)hydroxy-(2R)benzylhexanoyl)-L-leu-L-phe-amide; has a chemical structure of

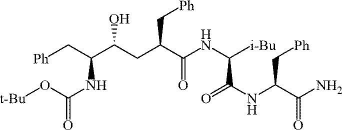

In one embodiments, the Notch4 modulating agent (e.g., agent that inhibits Notch4) is DAPT or L-685,458.

In some embodiments, the Notch4 modulating agent (e.g., agent that inhibits Notch4) is an antibody or antigen-binding fragment thereof, e.g., a Notch4 antibody or Notch4-binding fragment thereof. Various Notch4 antibodies known in the art can be used in the present invention. In one embodiment, the Notch4 modulating agent (e.g., agent that inhibits Notch4) is selected from the Notch4 antibodies shown in Table 1 below.

In some embodiments, the Notch4 modulating agent (e.g., agent that inhibits Notch4) is a humanized antibody. In some embodiments, the agent that inhibits Notch4 is a humanized version of an antibody selected from the antibodies shown in Table 1. One skilled in the art will be able to humanize an antibody using standard techniques.

TABLE 1

| | | | | |
|---|---|---|---|---|
| Clone ID | Vendor | Type | Species | Specificity |
| 07-189 | Upstate | Polyclonal | Rabbit | truncated, intracellular domain of Int3/Notch 4, possibly full-length Notch 4; does not cross-react with Notch 1, 2 or 3 |
| SAB4502023 | Milipore Sigma | Polyclonal | Rabbit | Anti-NOTCH4 (Cleaved-Val1432) antibody detects endogenous levels of fragment of activated NOTCH4 (Cleaved-Val1432) protein. |
| N5163 | Milipore Sigma | Polyclonal | Human, mouse, rat | synthetic peptide corresponding to residues 1506-1524 of human NOTCH4. This sequence is 89% identical in mouse and rat |
| HMN4-14 | Biolegend | Monoclonal | Mouse | reacts with mouse neurogenic locus notch homolog 4 (Notch4) a member of the Notch family of type 1 transmembrane proteins. |
| MHN4-2 | BD Pharmingen | Monoclonal | human | specifically binds to an extracellular domain of Notch4 |

Further examples of humanized Notch4 antibodies are known in the art, for example as described in US 9527921 (Antibody B), US 9969812 (Antibody B), U.S. Pat. No. 9676865, and International Patent Application WO 2018/039107 (e.g., antibody GLA-S4F18), the contents of each of which are incorporated herein by reference. In some embodiments, the Notch4 modulating agent (e.g., agent that inhibits Notch4) is an anti-Notch4 antibody as described in US 9527921, an antibody as described in US9969812B2, an antibody as described in US9676865B2, or an antibody as described in WO 2018/039107 (e.g., antibody GLA-S4F18). In some embodiments, the humanized Notch4 Ab comprises
(1) a heavy chain variable region comprising the amino acid sequence of SEQ ID NO: 3 and
(2) a light chain variable region comprising the amino acid sequence of SEQ ID NO: 4. In some embodiments, the humanized Notch4 Ab comprises (1) a heavy chain variable region comprising the amino acid sequence of SEQ ID NO: 5 and (2) a light chain variable region comprising the amino acid sequence of SEQ ID NO: 6.

TABLE 2

Exemplary sequences of humanized Notch4 antibodies

| | | |
|---|---|---|
| SEQ ID NO: | Antibody and region | Sequence |
| SEQ ID NO: 3 | Antibody B Heavy Chain Variable Region | EVQLVESGGGLVQPGGSLRLSCAASGFTFSSYGMSWVRQAPGKGLELVATINSNGGRTYYPDSVKGRFTISRDNSKNTLYLQMGSLKAEDMAVYYCARDQGFAYWGQGTLVTVSS |
| | (SEQ ID NO: 35 in US 9,969,812) | |
| SEQ ID NO: 4 | Antibody B Light Chain Variable Region | EIVMTQSPATLSVSPGERATLSCKASQDVGTAVAWYQQKPGQAPRLLIYWASTRHTGIPARFSGSGSGTEFTLTISSLQSEDFAVYYCQQYSSYPWTFGQGTKVEIK |
| | (SEQ ID NO: 45 in US 9,969,812) | |
| SEQ ID NO: 5 | GLA-S4F18 Heavy Chain Variable Region | QVQLVESGGGVVQPGRSLRLSCAASGFTFSSYGMHWVRQAPGEGLEWVATIPASGDNTYYADSVEGRFTISRDNSENTLYLQMNSLRAEDTAVYYCAKGAYKGYYYYIWWDVWGQGTMVTVSS |
| | (SEQ ID NO: 87 in WO 2018/0389107) | |
| SEQ ID NO: 6 | GLA-S4F18 Light Chain Variable Region | QSALTQPASVSGSPGQSITISCTGTSSDVGGYNYVSWYQQHPGEAPKLMIYEVSKRPSGVSNRFSGSKSGNTASLTISGLQAEDEADYYCSSYTRRSQRVFGGGTKLTVL |
| | (SEQ ID NO: 88 in WO 2018/0389107) | |

In one embodiment, the Notch4 modulating agent (e.g., agent that inhibits Notch4) is a Notch4 fusion protein, e.g., a Notch fusion protein as described in US 2016-0115217 A1, the contents of which is incorporated herein by reference.

### Identifying a Subject at Risk of Having Acute Respiratory Distress Syndrome (ARDS)

One aspect of the invention describe herein provides a method for identifying a subject at risk of developing acute respiratory distress syndrome (ARDS) comprising (a) obtaining a biological sample from the subject; (b) measuring the level of Notch4 in a population of candidate cell; (c) identifying a subject as being at risk of developing ARDS when the level of Notch is increased as compared to a reference level; and (d) administering a Notch4 modulating agent (e.g., an agent that inhibits Notch4) to a subject identified as being at risk of developing ARDS.

In one embodiment, the subject has been diagnosed as having COVID-19 prior to obtaining a biological sample.

In one embodiment, the method further comprises the step of, prior to obtaining a biological sample, diagnosing a subject as having COVID-19. In one embodiment of any aspect, the method further comprises the step of, prior to obtaining a biological sample, receiving the results of an assay that diagnoses a subject as having COVID-19.

In one embodiment, the level of Notch4 is increased at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, or more as compared to the reference level, or at least 10%, at least 20%, at least 30%, at least 40%, at least 50%, at least 60%, at least 70%, at least 80%, at least 90%, or at least 99% or more as compared to the reference level. The reference level can be the level of Notch4 in a sample obtained from a healthy subject, e.g., a subject who is not at risk of ARDS.

In one embodiment, the levels of Notch4 are measured in vitro or ex vivo. The levels of Notch4 in the sample can be measured using standard techniques, e.g., FACS analysis, or immunofluorescence. Protein and mRNA levels of Notch4 can be assessed using western blotting or PCR-based assays, respectively, as described herein.

In one embodiment, the biological sample is a blood sample, a peripheral blood sample, a sputum sample, a lung tissue sample, a lung biopsy sample, or a bronchial lavage sample. In one embodiment, the biological sample is any sample that contains alveolar macrophages. In one embodiment, the biological sample is taken from a subject that has previously been diagnosed with a coronavirus infectious disease (e.g., COVID-19) or ARDS. In one embodiment, the biological sample is taken from a subject that has previously been diagnosed with and treated for COVID-19 or ARDS. In one embodiment, the biological sample is taken from a subject that has not been diagnosed with COVID-19 or ARDS. Methods for collecting samples from a subject are known in art and can be performed by a skilled person.

### Administration

In some embodiments, the methods described herein relate to treating a subject having, diagnosed as having, or at risk of developing a coronavirus infectious disease (e.g., COVID-19) or acute respiratory distress syndrome (ARDS) comprising administering a Notch4 modulating agent (e.g., an agent that inhibits Notch4) as described herein. Subjects having or at risk of developing coronavirus infections disease (e.g., COVID-19) or ARDS can be identified by a physician using current methods of diagnosing a condition. Symptoms and/or complications of COVID-19, which characterize these disease and aid in diagnosis are well known in the art and include but are not limited to, persistent dry cough, trouble breathing, fever, loss of taste and/or smell, and gastrointestinal distress. Tests that may aid in a diagnosis of, e.g., COVID-19, include but is not limited nasopharyngeal swab that detects the RNA sequence specific to the coronavirus that causes COVID-19 (i.e., SARS-CoV-2). Symptoms and/or complications of acute respiratory distress syndrome are known in the art and include, but are not limited to, shortness of breath, rapid breathing or taking a lot of rapid and shallow breaths, rapid heart rate, coughing that produces phlegm, blue fingernails or blue tone to the skin or lops, fatigue, fever, and cracking sound in the lungs. Risk factors for ARDS are known in the art and include, but are not limited to, direct lung injury, system illness, injuries (including, e.g., from prolonged mechanical ventilation), and sepsis.

The agents described herein (e.g., a Notch4 modulating agent (e.g., an agent that inhibits Notch4)) can be administered to a subject having or diagnosed as having a coronavirus infectious disease (e.g., COVID-19) or ARDS. In some embodiments, the methods described herein comprise administering an effective amount of an agent to a subject in order to alleviate at least one symptom of a disease, e.g., COVID-19 or ARDS. As used herein, “alleviating at least one symptom of a disease” is ameliorating any condition or symptom associated with that disease. E.g., in the case of COVID-19 symptoms such as, e.g., persistent dry cough, trouble breathing, fever, loss of taste and/or smell, and gastrointestinal distress. As compared with an equivalent untreated control, such reduction is by at least 5%, 10%, 20%, 40%, 50%, 60%, 80%, 90%, 95%, 99% or more as measured by any standard technique. A variety of means for administering the agents described herein to subjects are known to those of skill in the art. In one embodiment, the agent is administered systemically or locally (e.g., to the lungs). In one embodiment, the agent is administered intravenously. In one embodiment, the agent is administered continuously, in intervals, or sporadically. The route of administration of the agent will be optimized for the type of agent being delivered (e.g., an antibody, a small molecule, an RNAi), and can be determined by a skilled practitioner.

In one embodiment, the agent, or compositions comprising an agent is administered through inhalation.

The term “effective amount” as used herein refers to the amount of an agent (e.g., a Notch4 modulating agent (e.g., an agent that inhibits Notch4)) can be administered to a subject having or diagnosed as having a coronavirus infectious disease (e.g., COVID-19) or ARDS needed to alleviate at least one or more symptom of the coronavirus infectious disease (e.g., COVID-19) or ARDS. The term “therapeutically effective amount” therefore refers to an amount of an agent that is sufficient to provide, e.g., a particular protective effect (e.g., anti-COVID-19 effect or protective effect against ARDS) when administered to a typical subject. An effective amount as used herein, in various contexts, would also include an amount of an agent sufficient to delay the development of a symptom of coronavirus infectious disease (e.g., COVID-19) or ARDS, alter the course of a symptom of the coronavirus infections disease (e.g., COVID-19 (e.g., slowing the progression of loss of lung function, inappropriate breathing, or ARDS)) or ARDS, or reverse a symptom of, e.g., (e.g., improve lung function or breathing). Thus, it is not generally practicable to specify an exact “effective amount”. However, for any given case, an appropriate “effective amount” can be determined by one of ordinary skill in the art using only routine experimentation.

In one embodiment, the agent is administered continuously (e.g., at constant levels over a period of time). Continuous administration of an agent can be achieved, e.g., by epidermal patches, continuous release formulations, or on-body injectors.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring neurological function, or blood work, among others. The dosage can be determined by a physician and adjusted, as necessary, to suit observed effects of the treatment.

### Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

The dosage of the agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

Typically, the dosage ranges for an agent, e.g., a small molecule, are between 0.001 mg/kg body weight to 5 g/kg body weight, inclusive. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 µg/kg body weight to 30 µg/kg body weight. Alternatively, the dose range will be titrated to maintain serum levels between 5 µg/mL and 30 µg/mL.

### Combinational Therapy

In one embodiment, the agent described herein is used as a monotherapy. In one embodiment, the agents described herein can be used in combination with other known agents and therapies for a coronavirus infectious disease (e.g., COVID-19) or ARDS. Administered “in combination,” as used herein, means that two (or more) different treatments are delivered to the subject during the course of the subject’s affliction with the disorder, e.g., the two or more treatments are delivered after the subject has been diagnosed with the disorder or disease (e.g., COVID-19) or ARDS and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agents described herein and the at least one additional therapy can be administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

In one embodiment, the additional therapeutic is an anti-viral. Exemplary anti-virals include, but are not limited to, Abacavir, Acyclovir (Aciclovir), Adefovir, Amantadine, Ampligen, Amprenavir (Agenerase), Arbidol, Atazanavir, Atripla, Balavir, Baloxavir marboxil (Xofluza®), Biktarvy Boceprevir (Victrelis®), Cidofovir, Cobicistat (Tybost®), Combivir (fixed dose drug), Daclatasvir (Daklinza®), Darunavir, Delavirdine, Descovy, Didanosine, Docosanol, Dolutegravir, Doravirine (Pifeltro®), Ecoliever, Edoxudine, Efavirenz, Elvitegravir, Emtricitabine, Enfuvirtide, Entecavir, Etravirine (Intelence®), Famciclovir, Fomivirsen, Fosamprenavir, Foscarnet, Fosfonet, Fusion inhibitor, Ganciclovir (Cytovene®), Ibacitabine, Ibalizumab (Trogarzo®), Idoxuridine, Imiquimod, Imunovir, Indinavir, Inosine, Integrase inhibitor, Interferon type I, Interferon type II, Interferon type III, Interferon, Lamivudine, Letermovir (Prevymis®), Lopinavir, Loviride, Maraviroc, Methisazone, Moroxydine, Nelfinavir, Nevirapine, Nexavir®, Nitazoxanide, Norvir, Nucleoside analogues, Oseltamivir (Tamiflu®), Peginterferon alfa-2a, Peginterferon alfa-2b, Penciclovir, Peramivir (Rapivab®), Pleconaril, Podophyllotoxin, Protease inhibitor (pharmacology), Pyramidine, Raltegravir, Remdesivir, Reverse transcriptase inhibitor, Ribavirin, Rilpivirine (Edurant®), Rimantadine, Ritonavir, Saquinavir, Simeprevir (Olysio®), Sofosbuvir, Stavudine, Synergistic enhancer (antiretroviral), Telaprevir, Telbivudine (Tyzeka®), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza®), and Zidovudine.

In one embodiment, the additional therapeutic is plasma obtained from a subject that was diagnosed as having COVID-19 and has recovered from. The plasma will contain antibodies that are useful in fighting the infection caused by COVID-19.

When administered in combination, the agent, or composition comprising the agent, and the additional therapeutic (e.g., second or third therapeutic), or all, can be administered in an amount or dose that is higher, lower or the same as the amount or dosage of each used individually, e.g., as a monotherapy. In certain embodiments, the administered amount or dosage of the agent, the additional therapeutic (e.g., second or third therapeutic), or all, is lower (e.g., at least 20%, at least 30%, at least 40%, or at least 50%) than the amount or dosage of each used individually. In other embodiments, the amount or dosage of agent, the additional therapeutic (e.g., second or third therapeutic), or all, that results in a desired effect (e.g., treatment of COVID-19 or ARDS) is lower (e.g., at least 20%, at least 30%, at least 40%, or at least 50% lower) than the amount or dosage of each individually required to achieve the same therapeutic effect.

### Parenteral Dosage Forms

Parenteral dosage forms of an agents described herein can be administered to a subject by various routes, including, but not limited to, subcutaneous, intravenous (including bolus injection), intramuscular, and intraarterial. Since administration of parenteral dosage forms typically bypasses the patient’s natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer’s injection, dextrose Injection, dextrose and sodium chloride injection, and lactated Ringer’s injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Aerosol Formulations

A composition comprising a Notch4 modulating agent (e.g., an agent that inhibits Notch4) can be administered directly to the airways of a subject in the form of an aerosol or by nebulization. For use as aerosols, a Notch4 modulating agent (e.g., an agent that inhibits Notch4) in solution or suspension may be packaged in a pressurized aerosol container together with suitable propellants, for example, hydrocarbon propellants like propane, butane, or isobutane with conventional adjuvants. A Notch4 modulating agent (e.g., An agent that inhibits Notch4) can also be administered in a non-pressurized form such as in a nebulizer or atomizer.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means therefore, including by using many nebulizers known and marketed today. For example, an AEROMIST pneumatic nebulizer available from Inhalation Plastic, Inc. of Niles, Ill. When the active ingredients are adapted to be administered, either together or individually, via nebulizer(s) they can be in the form of a nebulized aqueous suspension or solution, with or without a suitable pH or tonicity adjustment, either as a unit dose or multidose device.

As is well known, any suitable gas can be used to apply pressure during the nebulization, with preferred gases to date being those which are chemically inert to a modulator of a Notch4 modulating agent (e.g., an agent that inhibits Notch4). Exemplary gases including, but are not limited to, nitrogen, argon or helium can be used to high advantage.

In some embodiments, a Notch4 modulating agent (e.g., an agent that inhibits Notch4) can also be administered directly to the airways in the form of a dry powder. For use as a dry powder, a GHK tripeptide can be administered by use of an inhaler. Exemplary inhalers include metered dose inhalers and dry powdered inhalers.

A metered dose inhaler or “MDI” is a pressure resistant canister or container filled with a product such as a pharmaceutical composition dissolved in a liquefied propellant or micronized particles suspended in a liquefied propellant. The propellants which can be used include chlorofluorocarbons, hydrocarbons or hydrofluoroalkanes. Especially preferred propellants are P134a (tetrafluoroethane) and P227 (heptafluoropropane) each of which may be used alone or in combination. They are optionally used in combination with one or more other propellants and/or one or more surfactants and/or one or more other excipients, for example ethanol, a lubricant, an anti- oxidant and/or a stabilizing agent. The correct dosage of the composition is delivered to the patient.

A dry powder inhaler (i.e. Turbuhaler (Astra AB)) is a system operable with a source of pressurized air to produce dry powder particles of a pharmaceutical composition that is compacted into a very small volume.

Dry powder aerosols for inhalation therapy are generally produced with mean diameters primarily in the range of <5 µm. As the diameter of particles exceeds 3 µm, there is increasingly less phagocytosis by macrophages. However, increasing the particle size also has been found to minimize the probability of particles (possessing standard mass density) entering the airways and acini due to excessive deposition in the oropharyngeal or nasal regions.

Suitable powder compositions include, by way of illustration, powdered preparations of a Notch4 modulating agent (e.g., an agent that inhibits Notch4) thoroughly intermixed with lactose, or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants, and co-solvents and may be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

### Controlled and Delayed Release Dosage Forms

In some embodiments, an agent is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)′s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any agent described herein. Examples include, but are not limited to, those described in U.S. Pat. Nos.: 3,845,770; 3,916,899; 3,536,809; 3,598,123; 4,008,719; 5674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

### Efficacy

The efficacy of an agents described herein, e.g., for the treatment of a coronavirus infectious disease (e.g., COVID-19), can be determined by the skilled practitioner. However, a treatment is considered “effective treatment,” as the term is used herein, if one or more of the signs or symptoms of, e.g., COVID-19, are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., increased lung function, reduced fever, restored normal breathing. Efficacy can also be measured by a failure of an individual to worsen as assessed by hospitalization, or need for medical interventions (i.e., progression of diminished lung function, complications with breathing, ARDS). Methods of measuring these indicators are known to those of skill in the art and/or are described herein.

Efficacy can be assessed in animal models of a condition described herein, for example, a mouse model or an appropriate animal model of COVID-19, as the case may be. When using an experimental animal model, efficacy of treatment is evidenced when a statistically significant change in a marker is observed, e.g., decreased airway inflammation, increased lung function, restored normal breathing.

Efficacy of a Notch4 modulating agent (e.g., an agent that inhibits Notch4) can additionally be assessed using methods described herein.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway’s Immunobiology, Kenneth Murphy, Allan Mowat, Casey Weaver (eds.), W. W. Norton & Company, 2016 (ISBN 0815345054, 978-0815345053); Lewin’s Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

### Summary for Examples 1-5

A cardinal feature of COVID-19 is lung inflammation and respiratory failure. In a prospective multi-country cohort of COVID-19 patients, it was found that increased Notch4 expression on circulating regulatory T (Treg) cells was associated with disease severity, predicted mortality, and declined upon recovery. Deletion of Notch4 in Treg cells in conventional and humanized mice normalized the dysregulated innate immunity and rescued disease morbidity and mortality induced by a synthetic analogue of viral RNA or by influenza H1N1 virus. Mechanistically, Notch4 suppressed the induction by interleukin-18 of amphiregulin, a cytokine necessary for tissue repair. Amphiregulin declined in COVID-19 subjects as a function of disease severity and Notch4 expression. Thus, Notch4 expression on Treg cells dynamically restrains amphiregulin-dependent tissue repair to promote severe lung inflammation, with therapeutic implications for COVID-19 and related infections.

The 2020 pandemic caused by the severe acute respiratory syndrome coronavirus 2 (SARS-CoV-2) virus has resulted in massive morbidity and mortality figures both within the US and globally (Cucinotta and Vanelli, 2020). While many infected subjects are either asymptomatic or have a mild form of the disease, a subset suffers a more severe disease with pneumonia and marked hypoxia, leading to acute respiratory distress syndrome (Berlin et al., 2020; Richardson et al., 2020; Zhou et al., 2020a). In many of those patients, the disease proved fatal despite intensive respiratory support. Uncontrolled activation of the immune response, leading to a cytokine storm, is a key risk factor for mortality (Henderson et al., 2020; Lucas et al., 2020; Vabret et al., 2020). The molecular mechanisms governing lung disease severity are not well understood yet. Notably, innate immune cell hyperactivation plays a critical role in the pathogenesis of severe COVID-19 (Del Valle et al., 2020; Vabret et al., 2020; Zhou et al., 2020b), a finding also recapitulated in emerging mouse models (Winkler et al., 2020). Among the innate immune cytokines, interleukin (IL)-6 has been proposed to play a pathogenic role by virtue of its increased serum concentrations in severe COVID-19 subjects and reports of favorable clinical responses to anti-interleukin 6 receptor (IL-6R) monoclonal antibody (mAb) therapy (Copaescu et al., 2020; Investigators et al., 2021; Salama et al., 2021; Toniati et al., 2020; Xu et al., 2020). Other studies however have failed to confirm such a benefit (Hermine et al., 2021; Rosas et al., 2021; Salvarani et al., 2021; Veiga et al., 2021).

An IL-6-dependent pathway subverts lung regulatory T (Treg) cells to promote tissue inflammation in severe asthma by increasing Treg cell expression of the receptor Notch4 (Harb et al., 2020; Xia et al., 2018; Xia et al., 2015). In this setting, Notch4 acts via downstream pathways, including Hippo and Wnt, to disrupt Treg cell regulation of the T helper type 2 (Th2) and Th17 adaptive immune responses. In case studies, treatment of severe asthma patients with the anti-IL-6R mAb

The NOTCH4 locus is associated with critical illness in COVID-19 (Pairo-Castineira et al., 2021). Given this and the evidence supporting a pathogenic role for high levels of IL-6 in COVID-19, the impact of inducible Notch4 expression on Treg cells in lung viral infection, including COVID-19, was examined. It was found that increased Notch4 expression in COVID-19 subjects is a function of disease severity. In mouse models of respiratory viral infections, Notch4 enabled virus-induced tissue inflammation by mechanisms distinct from those involved in its regulation of adaptive immunity in allergic airway inflammation. Rather, Notch4 expression inhibited Treg cell-mediated regulation of innate immune responses and promotion of tissue repair. The protective function of Notch4 inhibition involved increased production by Treg cells of the epidermal growth factor-like cytokine amphiregulin, which plays a critical role in in mediating tissue repair by Treg cells in lung viral infections (Arpaia et al., 2015). These findings identify Notch4 as an effector of COVID-19 disease severity, and point to interventions along the Notch4-amphiregulin axis as a viable therapeutic strategy to restore immune regulation in severe viral respiratory infections including COVID-19.

### Example 1

### Treg Cell Notch4 Expression is Independently Predictive of Mortality From COVID-19

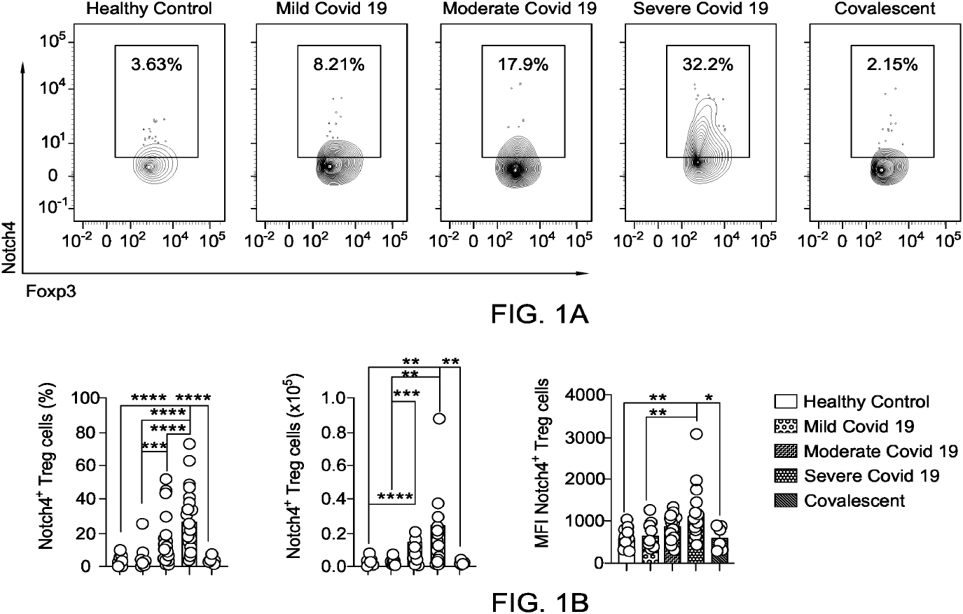

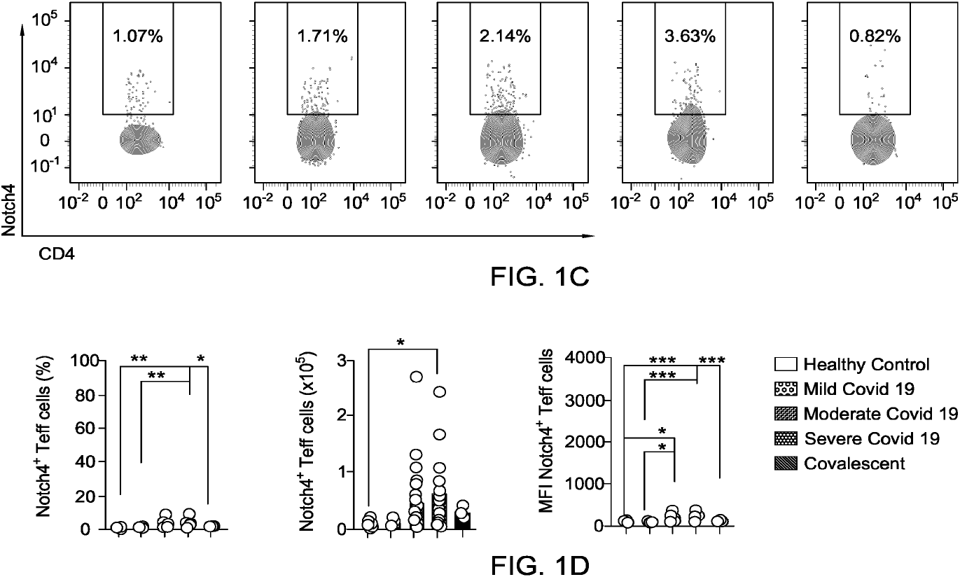

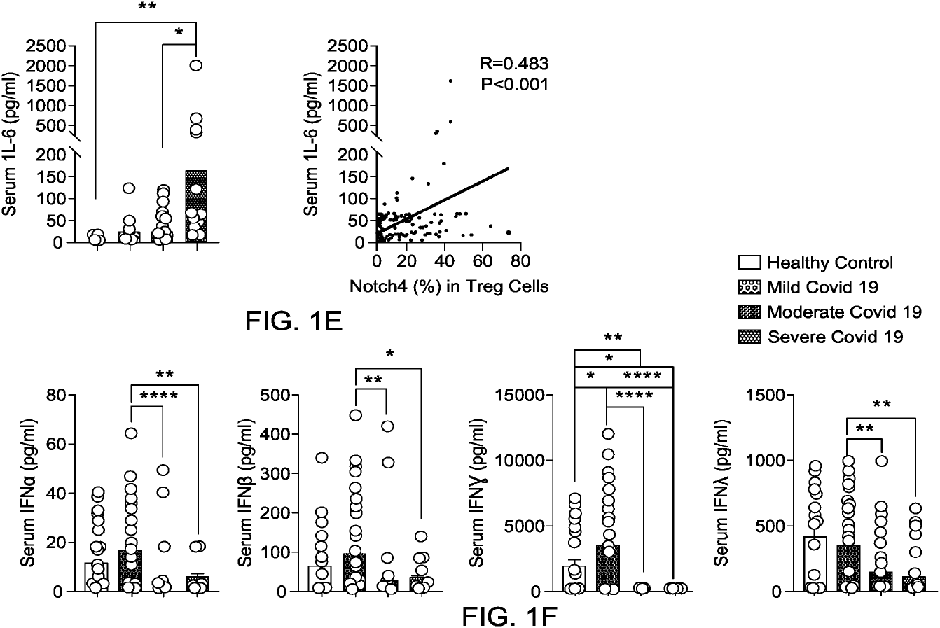

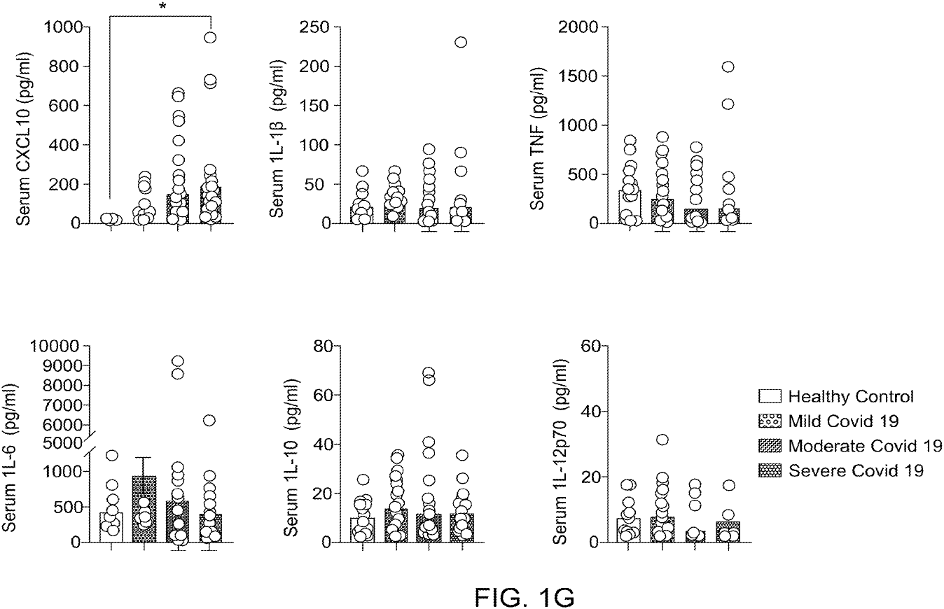

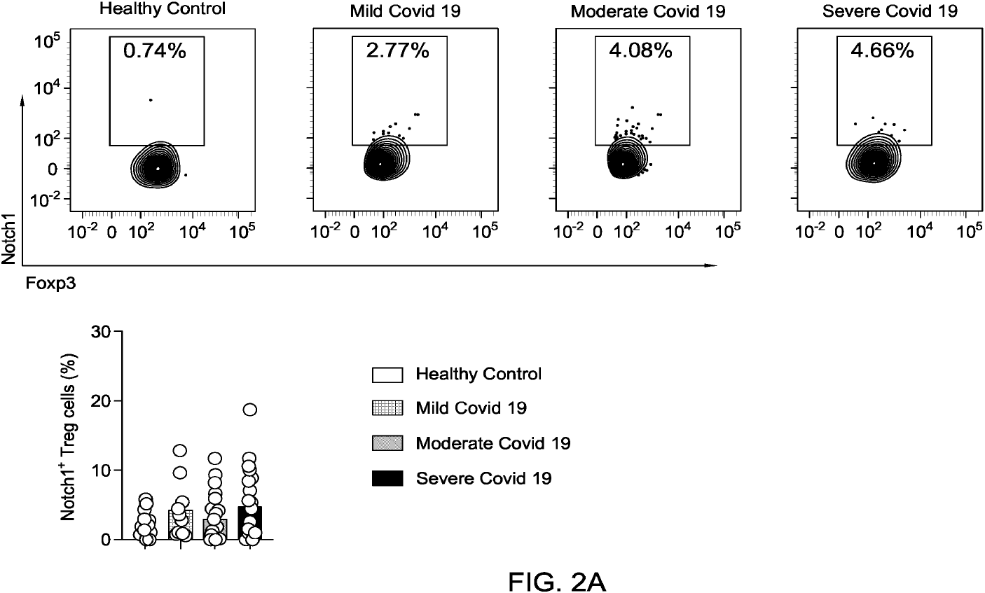

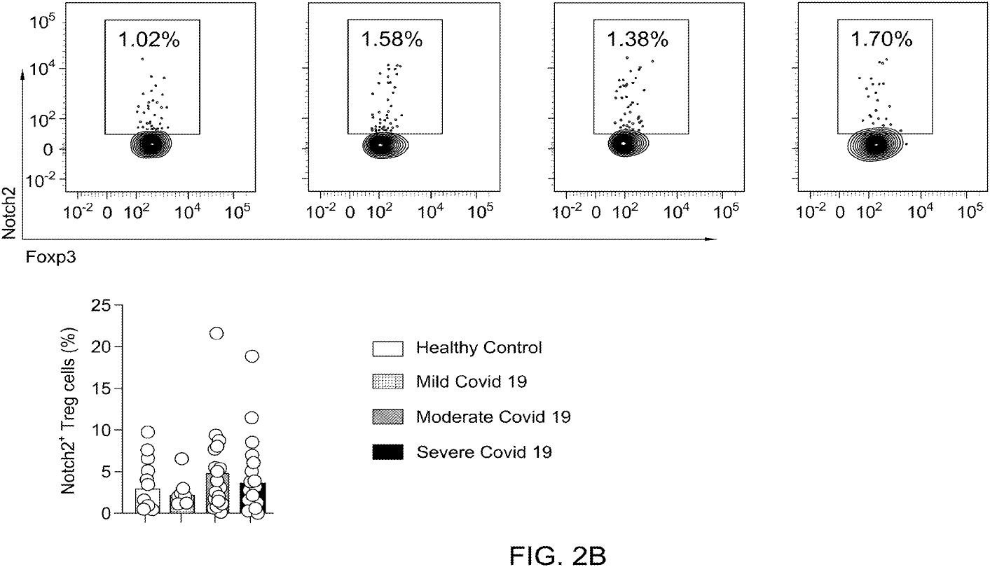

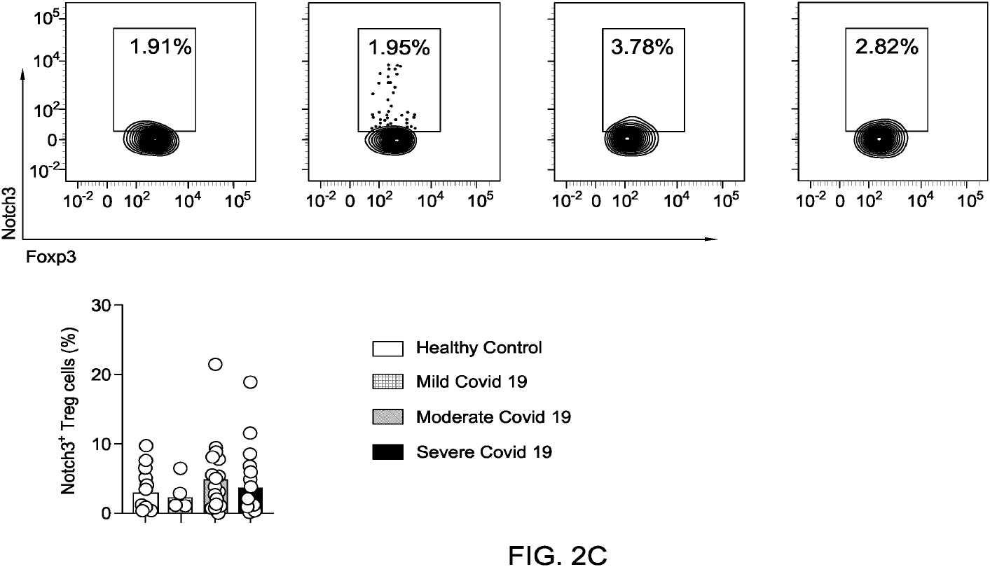

To determine the association between Notch4 and disease severity in COVID-19 patients, three cohorts of patients with COVID-19, as well as healthy controls were recruited from Boston, Massachusetts (n=38 and n=10, respectively), Genoa, Italy (n=44 and n=10) and Istanbul, Turkey (n=36 and n=20) (Table 2). The patients were segregated into three disease severity groups (mild, moderate and severe) based on the need for hospitalization and advanced respiratory support, as well as a convalescent group following criteria detailed in the Methods section. Older male patients with a history of malignancy, cardiac disease, or endocrine disease had more severe disease (Table 2). Frequencies, absolute numbers and mean fluorescence intensity (MFI) of Notch4 expression on peripheral blood CD4+CD25+Foxp3+ Treg cells of COVID-19 subjects in the different cohorts progressively increased as a function of disease severity and declined precipitously in the convalescent subjects (FIGS. 1A, 1B and Table 2). In contrast, there was a modest increase in the Notch4 expression on CD4+CD25-Foxp3- T effector (Teff) cells of subjects with moderate and severe disease, and otherwise minimal expression in the other subject groups (FIGS. 1C, 1D). Also, there was no difference in the expression of the other Notch receptors including Notch1, Notch2 and Notch3 on circulating Treg cells amongst subjects in the different study groups (FIGS. 2A-2C). In multivariate logistic regression adjusting for age, gender, comorbidities, glucocorticoid treatment, and serum IL-6 levels, each 1% increase in Notch4 expression in Treg cells was independently associated with a 1.046 higher odds of mortality (univariate odds ratio 1.055 [95% CI 1.008 - 1.090, p = 0.023; multivariate odds ratio 1.057 [95% CI 1.01 - 1.11], p = 0.017 (Table 2). Thus, for example, an increase in Treg cells expressing Notch4 by 10% in a 65-year-old male patient with a history of malignancy treated with steroids would lead to a rise in predicted mortality from 28.4% to 38.3%. Overall, serum IL-6 concentrations were positively correlated with Treg cell Notch4 expression (FIG. 1E). Moreover, the serum concentrations of interferon alpha (IFNα), IFNβ and IFNγ were decreased in the moderate and severe patients compared to mild patients (FIG. 1F) (Blanco-Melo et al., 2020; Hadjadj et al., 2020). In contrast, the serum concentrations of other cytokines including IL-1β, TNF, IL-8, IL-10, IL-12p70 were unchanged, while those of CXCL10 were slightly increased in the severe subjects (FIG. 1G).

### Example 2

### Treg Cell Notch4 Promotes Lung Tissue Inflammation Induced by Polyinosinic: Polycytidylic Acid (Poly I:C)

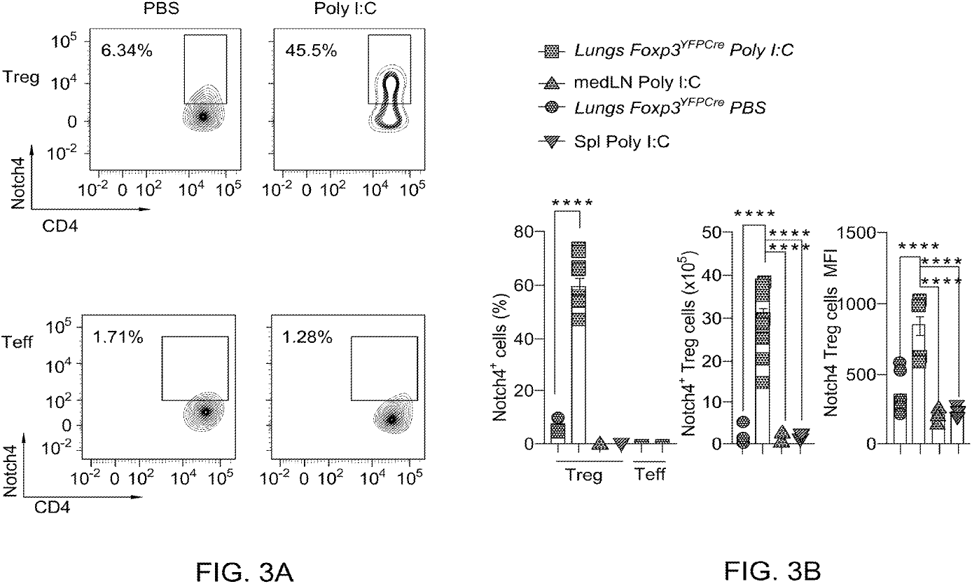

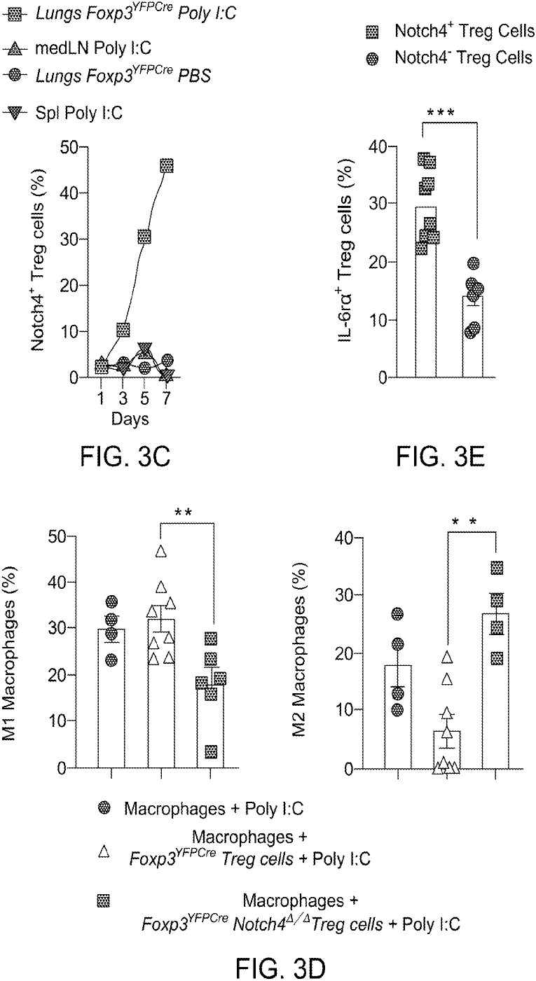

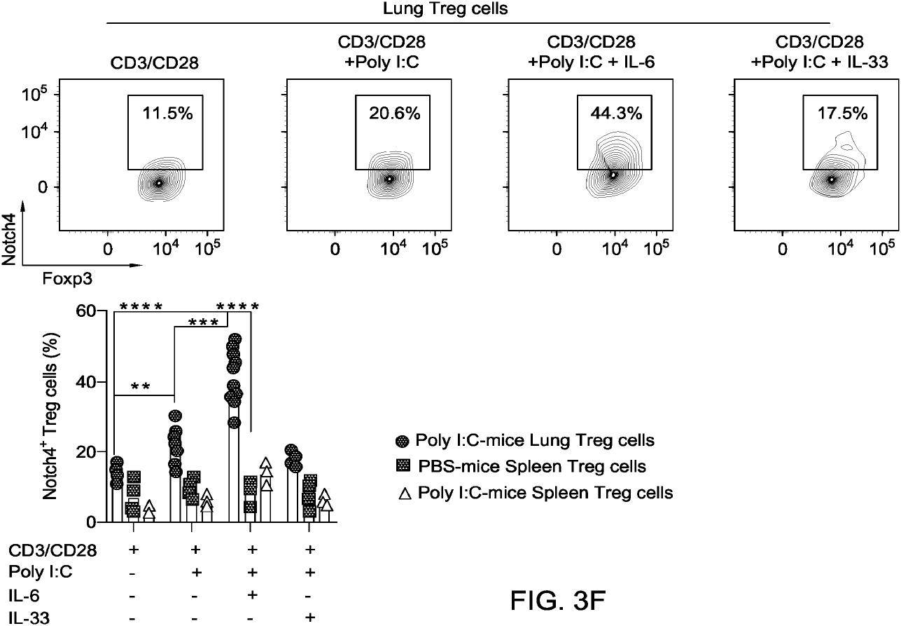

To investigate the mechanism by which Notch4 licenses lung tissue inflammation in viral respiratory infections, its role in Poly I:C-induced airway inflammation was first examined. Poly I:C stimulates Toll like receptor 3 and the downstream viral RNA sensors cytoplasmic retinoic acid-inducible gene I (RIG-I) and melanoma differentiation-associated protein 5 (MDA5), thus providing a proxy model of RNA viral infections (Broggi et al., 2020; Iwasaki and Pillai, 2014; Kato et al., 2006). Daily intra-tracheal instillation of Poly I:C in mice for 6 consecutive days resulted in a sharp progressive increase in the frequencies and absolute numbers of Notch4 expressing lung tissue Treg cells as well as the MFI of Notch4 on those cells (FIGS. 3A-3C). In contrast, minimal expression of Notch4 was noted on CD4+ Teff cells. Treg cell specific deletion of Notch4 using a Foxp3-driven Cre recombinase (Foxp3YFPCre) and floxed Notch4 allele (Foxp3YFPCreNotch4Δ/Δ) protected the mice from the weight loss induced by the Poly I:C treatment (data not shown).

Further analysis revealed that Notch4 deletion suppressed lung tissue neutrophil infiltration induced by Poly I:C treatment and reversed the increase in lung M1 macrophages while increasing the M2 macrophages (Data not shown). In vitro co-cultures of Poly I:C-treated macrophages with lung Treg cells derived from Poly I:C-treated mice revealed that Foxp3YFPCreNotch4Δ/Δ but not Foxp3YFPCre Treg cells reversed the M1 skewing and promoted M2 macrophage differentiation (FIG. 3D). These results confirmed a critical role for Treg cell Notch4 expression in the evolution of lung inflammation induced by Poly I:C.

### Example 3

### Specificity for Notch4’s Role in Promoting Lung Inflammation

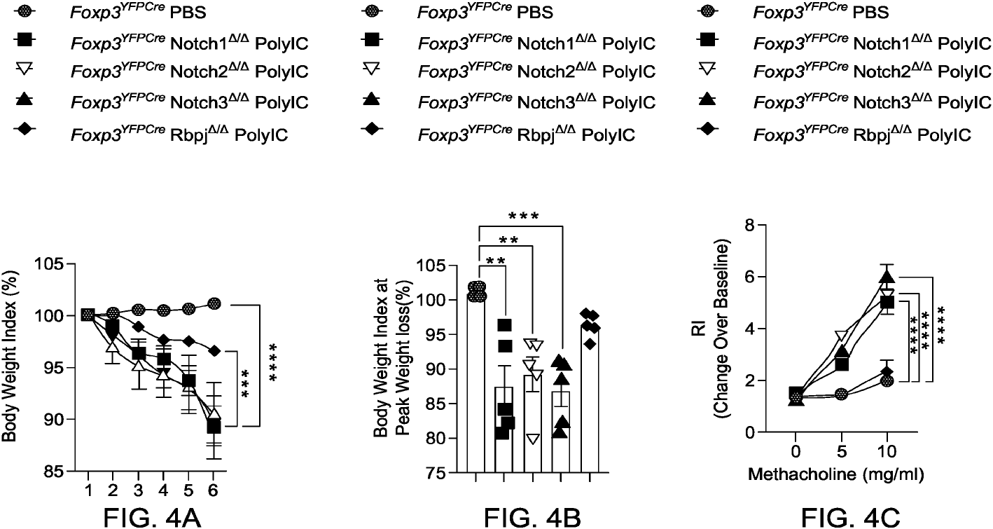

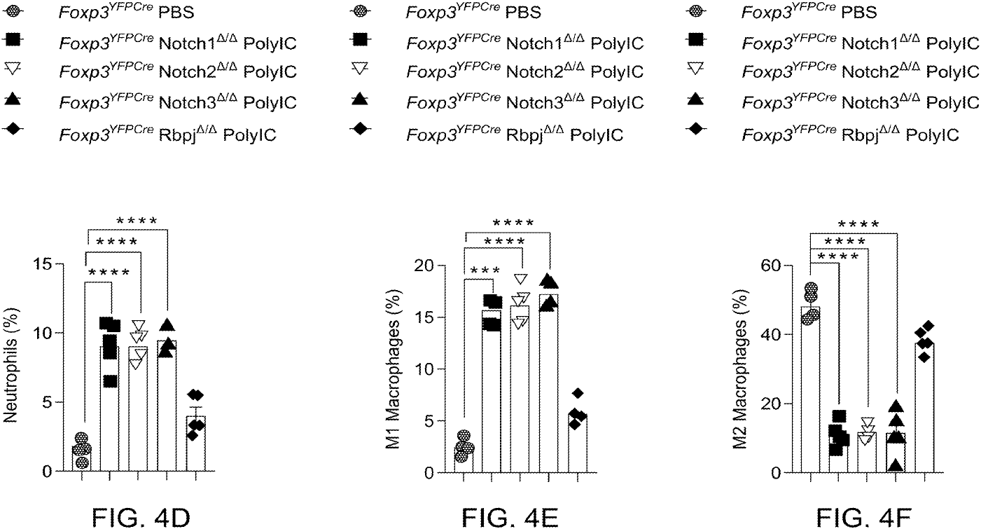

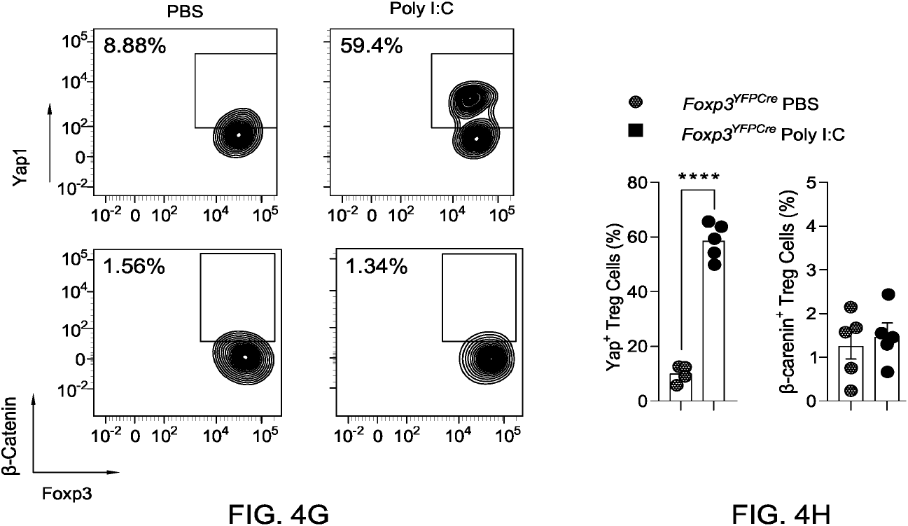

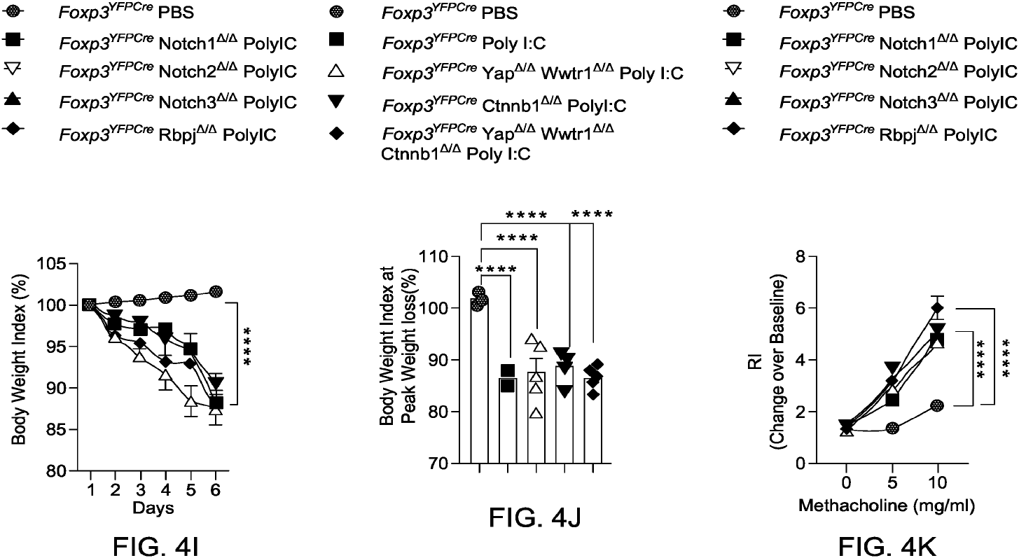

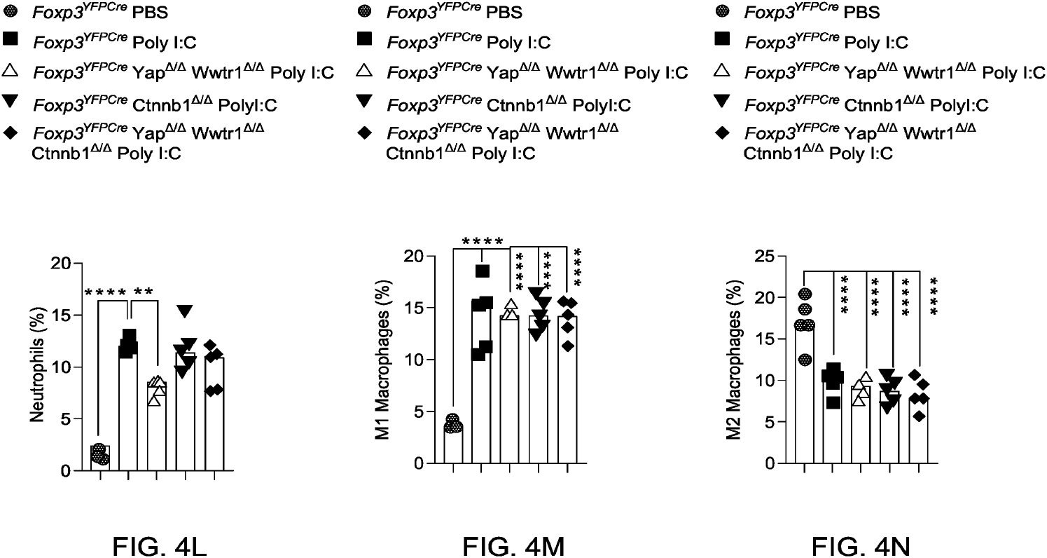

Different Notch receptors control Treg cell function (Charbonnier et al., 2015; Ostroukhova et al., 2006; Perumalsamy et al., 2012). However, the role of Notch4 in regulating lung tissue inflammation by Poly I:C was highly specific in that Treg cell-specific deletion of the other Notch genes, including Notch1, Notch2 and Notch3, failed to protect against tissue inflammation. In contrast, Treg cell-specific deletion of Rbpj, encoding the Notch canonical pathway transcription factor RBPJ, largely reproduced the protective effect of Notch4 deletion (FIGS. 4A-4F). Downstream of Notch4, the inventors’ previous studies have implicated the Hippo and Wnt pathways in Treg cell regulation of Th17 and Th2 responses in allergic airway inflammation, respectively (Harb et al., 2020). Poly I:C treatment potently induced the expression of the Hippo pathway effector Yap in lung tissue Treg cells, while the expression of Wnt pathway effector □-Catenin was unchanged (FIGS. 4G, 4H). Treg cell-specific deletion of Yap1 and Wwtr1, encoding the Hippo pathway effectors Yap and Taz, Ctnnb1, encoding the Wnt pathway effector □-Catenin, or all three genes failed to protect against Poly I:C-induced inflammation. These findings indicated that Notch4 regulation of the acute inflammatory responses to viral infection proceeds by distinct mechanisms (FIGS. 4I-4N).

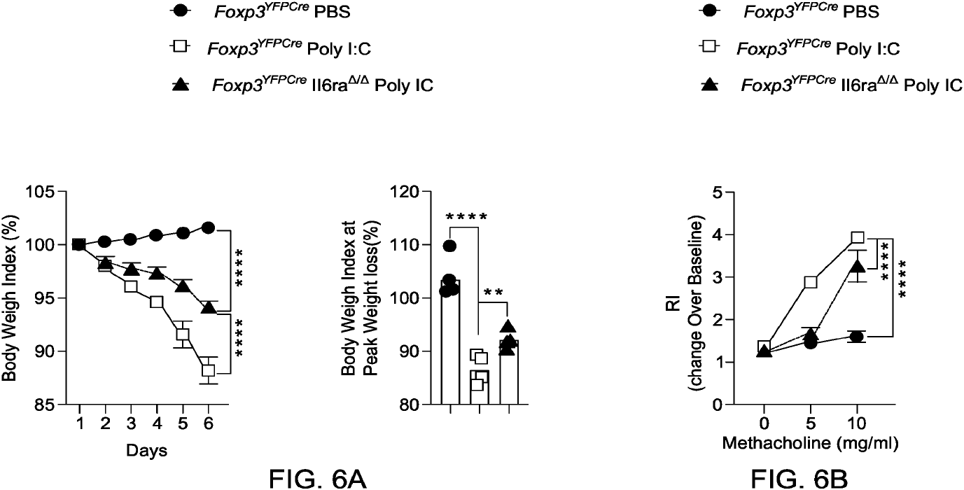

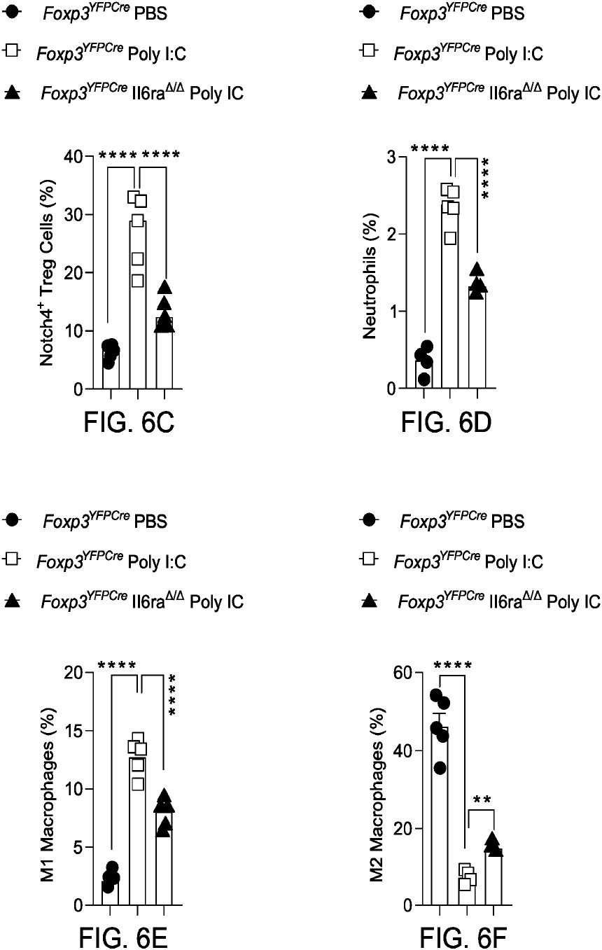

The inventors have previously identified a key role for IL-6 in upregulating Notch4 expression on Treg cells in asthmatic inflammation (Harb et al., 2020). Consistent with these results, Notch4+ lung Treg cells from Poly I:C-treated mice expressed higher levels of IL-6Rα chain as compared to their Notch4- counterparts (FIG. 3E). To further elucidate the mechanisms of Notch4 induction in Treg cells, in vitro studies were conducted to examine the capacity of Poly I:C and cytokine treatment to upregulate Notch4 expression in Treg cells isolated from PBS or Poly I:C-treated mice. Results showed that Poly I:C treatment of lung but not splenic Treg cells increased Notch4 expression in synergy with IL-6 but not IL-33 (FIG. 3F). In vivo, Treg cell-specific deletion of Il6ra attenuated the Poly I:C-induced Notch4 expression on lung Treg cells and the attendant tissue inflammatory response, including the decline in body weight, the increase in AHR, the influx of neutrophils and the alteration in macrophage populations (FIGS. 6B-6G). These results indicated that both IL-6 and additional signals contribute to Notch4 upregulation on lung Treg cells in Poly I:C-treated mice.

### Example 4

### Protection by Notch4 Antagonism is Amphiregulin-Dependent

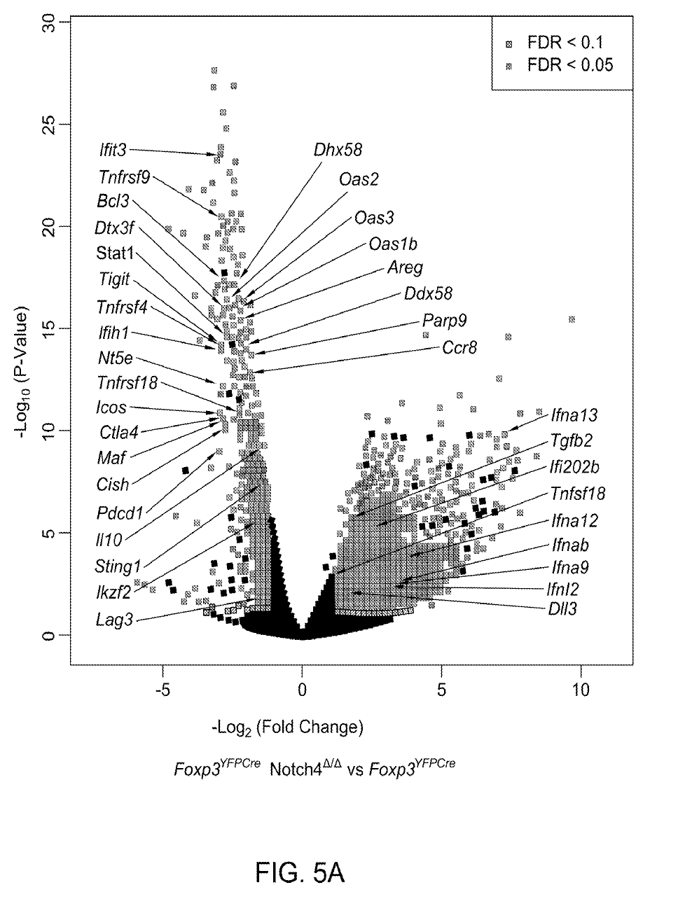

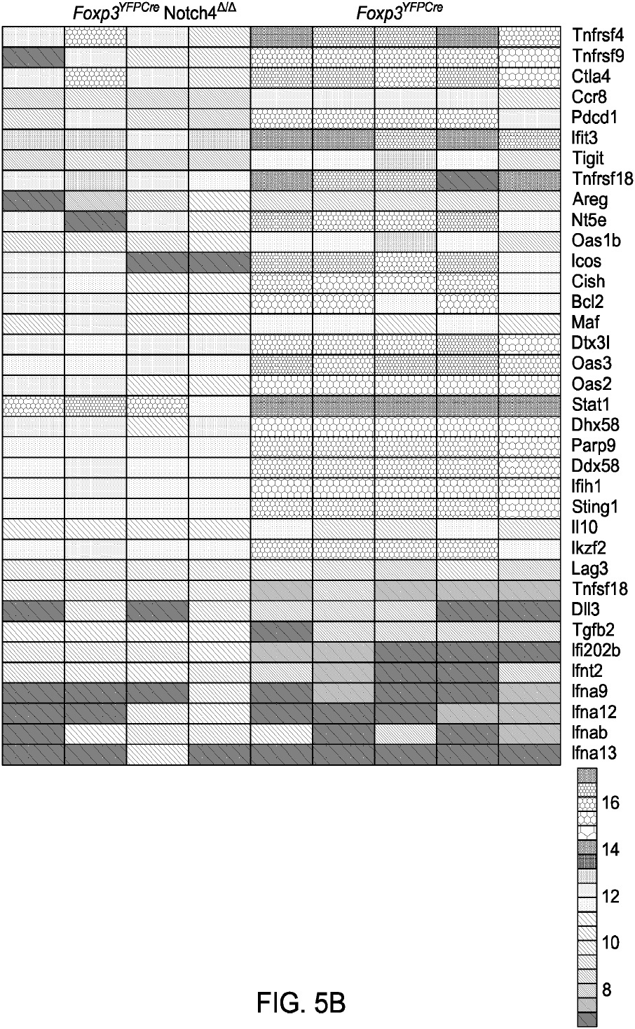

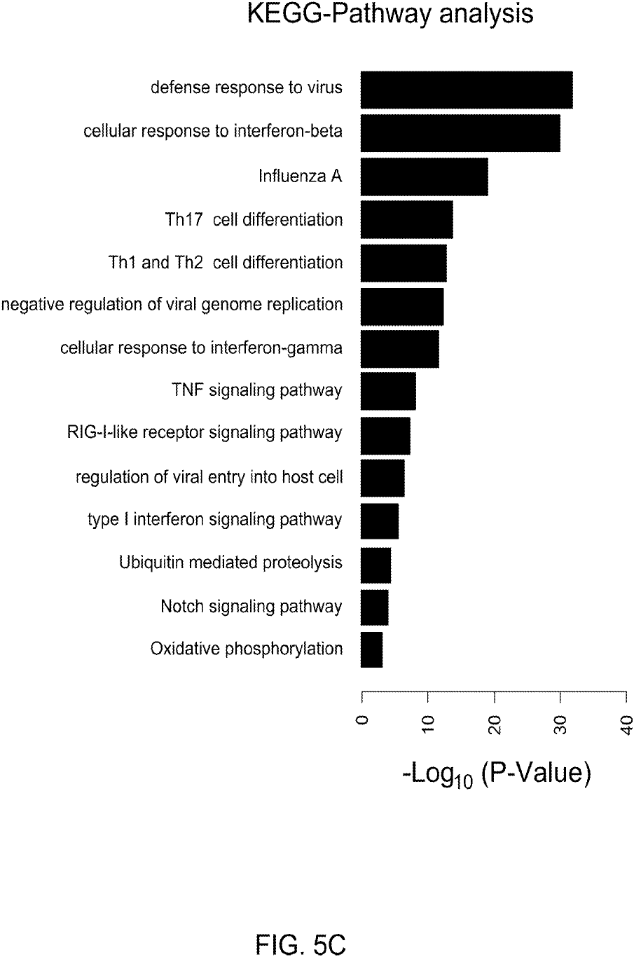

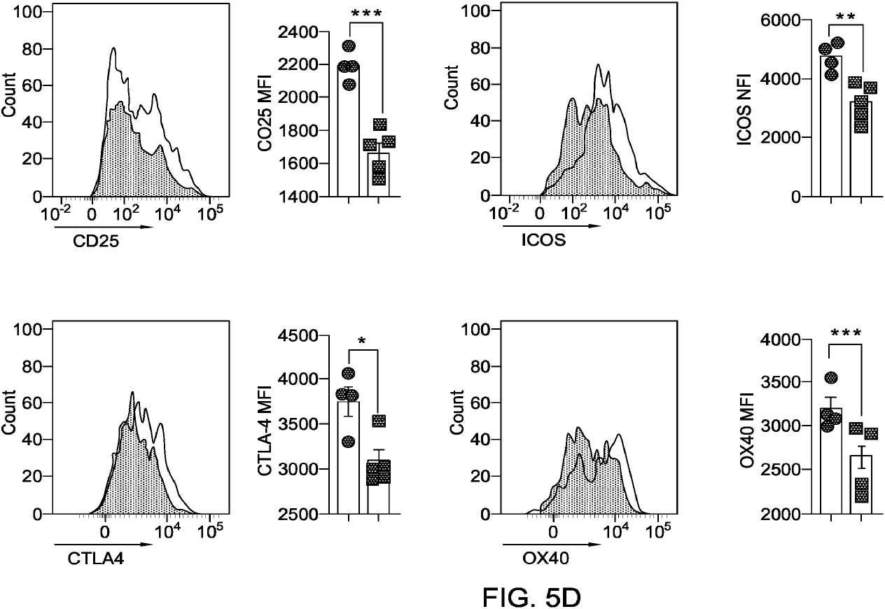

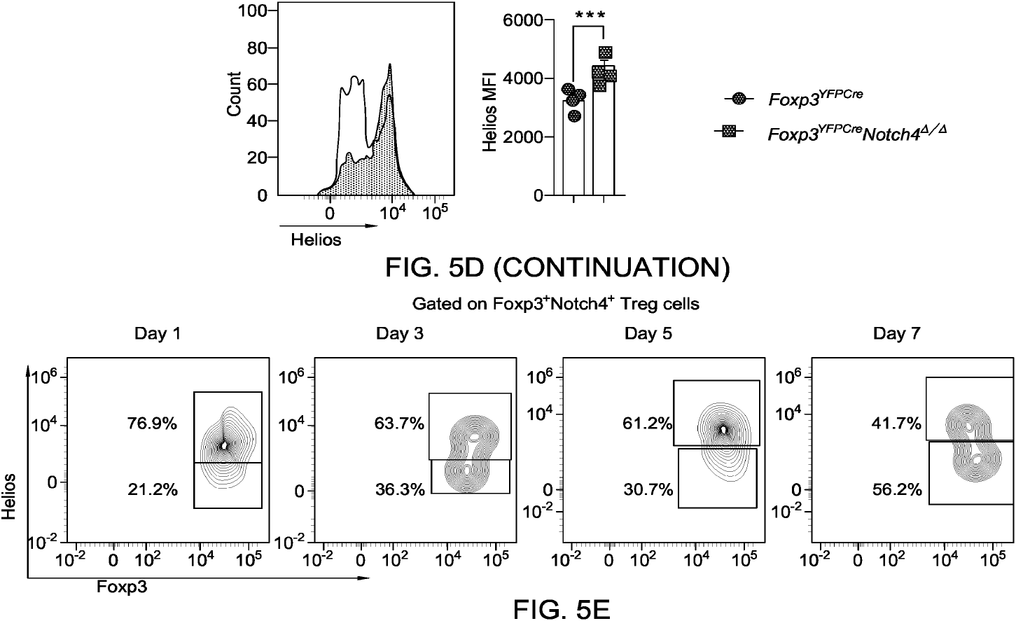

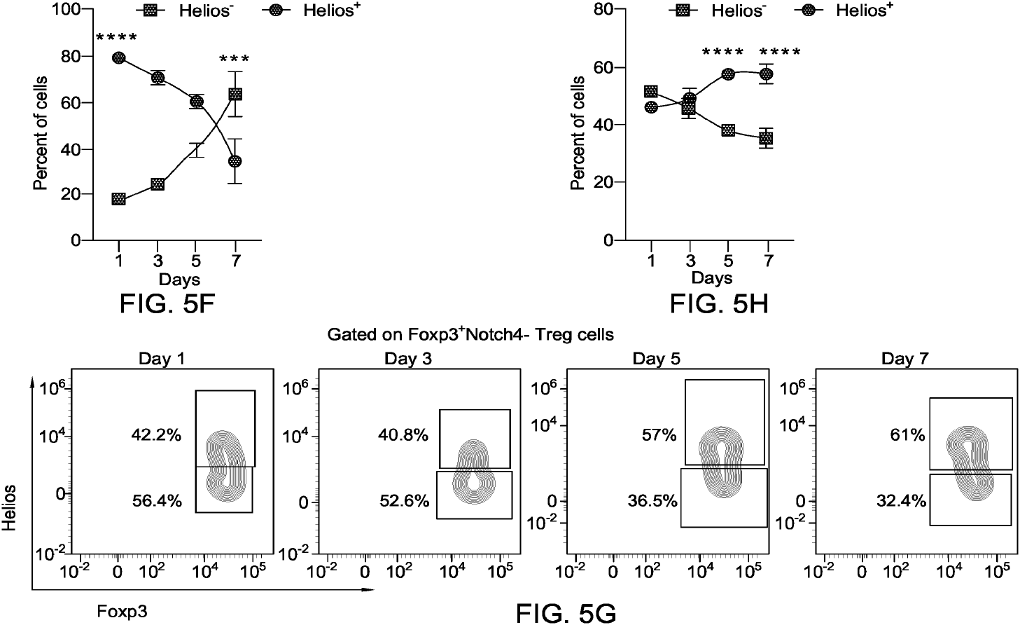

To better understand the mechanisms by which Notch4 depletion protected against lung inflammation, the transcriptomes of lung Treg cells of Foxp3YFPCre and Foxp3YFPCreNotch4Δ/Δ mice treated with Poly I:C for 6 days were analyzed. Results revealed that the lung Treg cells of Poly I:C treated Foxp3YFPCre mice exhibited an activated effector Treg cell signature, with upregulation of many canonical Treg cell transcripts including Ctla4, Ikzf2, Il10, Tigit and the TNF receptor superfamily members Tnfrsf4, Tnfrsf9 and Tnfrsf18 (Arpaia et al., 2015) (FIGS. 5A, 5B). Poly I:C treatment also imparted an exhausted T cell-like signature with increased Pdcd1, Icos, Lag3 and other related transcripts that may impair their suppressive function (Lowther et al., 2016). In contrast, Treg cells of Poly I:C-treated Foxp3YFPCreNotch4Δ/Δ mice increased several type I interferon genes. More broadly, key pathways enriched in Foxp3YFPCre versus Foxp3YFPCreNotch4Δ/Δ lung Treg cells included those involved in innate viral sensing and response, type I interferon signaling, TH cell differentiation and ubiquitin-mediated proteolysis (FIG. 5C). While the RNA and protein expression of several key canonical markers was concordantly increased in the Foxp3YFPCre lung Treg, exceptions included the transcription factor Helios, encoded by Ikzf2, whose expression was decreased despite increased transcript levels (FIG. 5D). Further analysis localized the loss of Helios expression to the Notch4+ fraction of the Foxp3YFPCre lung Treg, suggesting either an expansion of Helioslow Notch4+ induced Treg cells, similar to the inventors’ prior observation in the asthma model (Harb et al., 2020), or alternatively the loss of Helios expression leading to an increased potential for destabilization (Thornton et al., 2019) (FIG. 5E). The latter results indicated that Notch4 initiated a program of post-translational regulation by pathways such as ubiquitination and proteolysis, both of which were increased in the Treg cells of poly I:C-treated Foxp3YFPCre mice.

The function of Treg cells in lung tissue repair in H1N1 virus infection involves the production of epidermal growth factor-like cytokine amphiregulin (Arpaia et al., 2015). Treg cells are the main source of amphiregulin in the lung, while Treg cell-specific deletion of Areg, the gene encoding amphiregulin, worsens disease outcome (Arpaia et al., 2015). Analysis of lung Treg cells revealed that despite markedly increased Areg transcripts in Foxp3YFPCre as compared to Foxp3YFPCreNotch4Δ/Δ Treg cells following poly I:C treatment (data not shown), amphiregulin protein expression failed to increase in the Treg cells and BAL fluid of Foxp3YFPCre mice. Consistent with these results, amphiregulin expression was found differentially enriched in Notch4- versus Notch4+ lung Treg cell in Poly I:C treated mice (data not shown). Similar results were also found in lung Treg cells of influenza H1N1 virus-infected mice (data not shown).

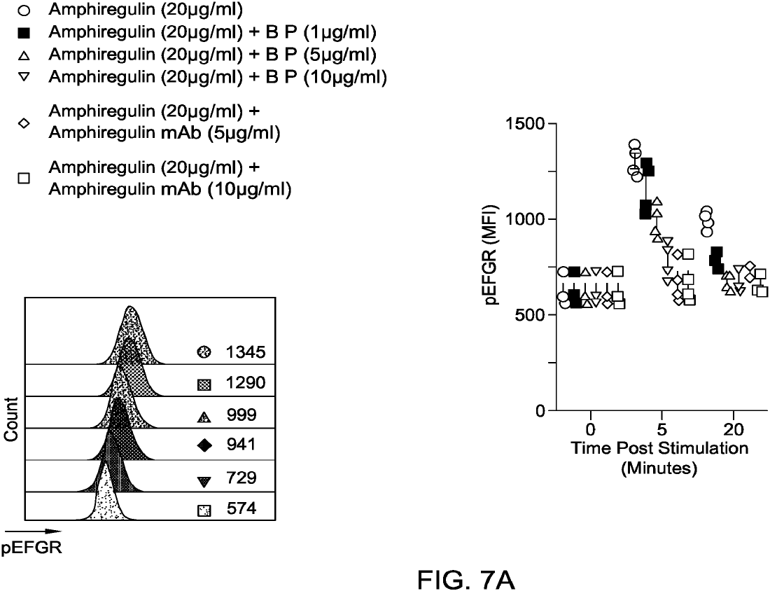

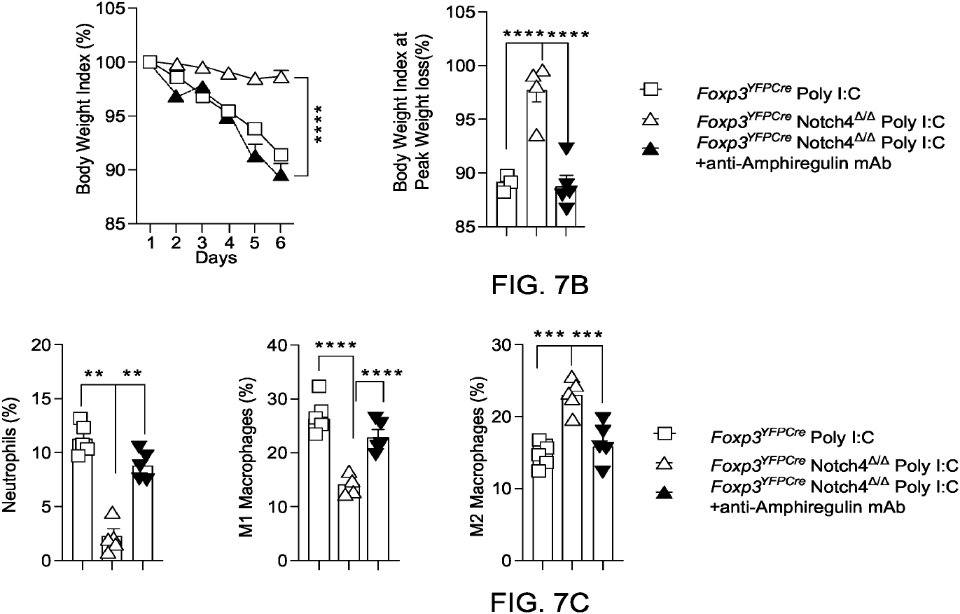

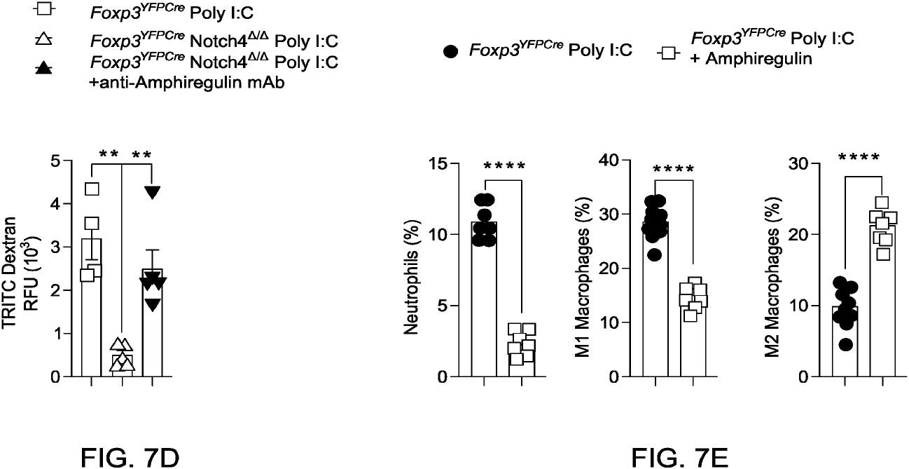

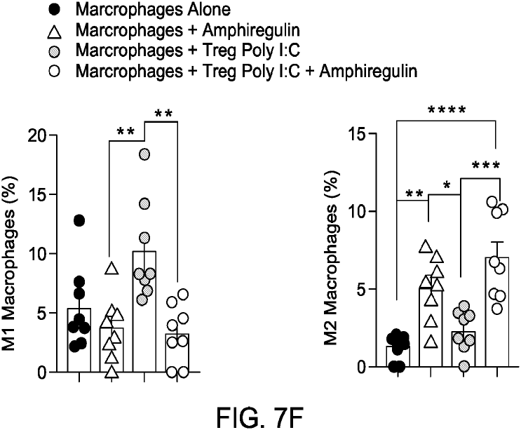

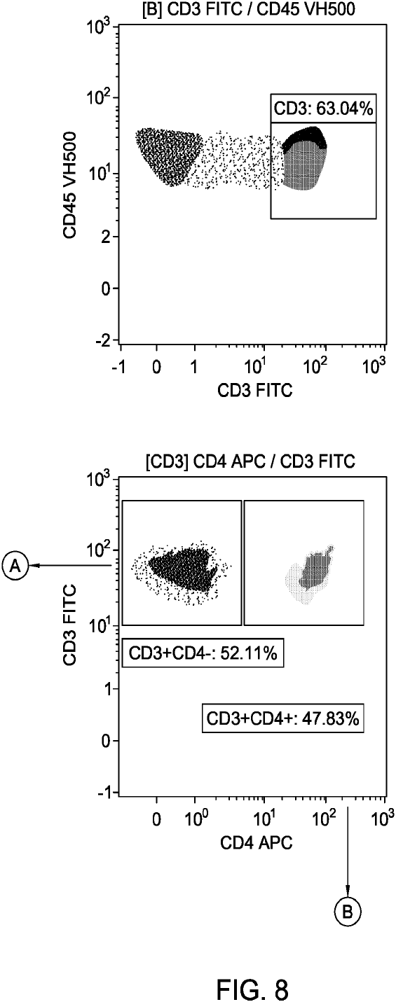

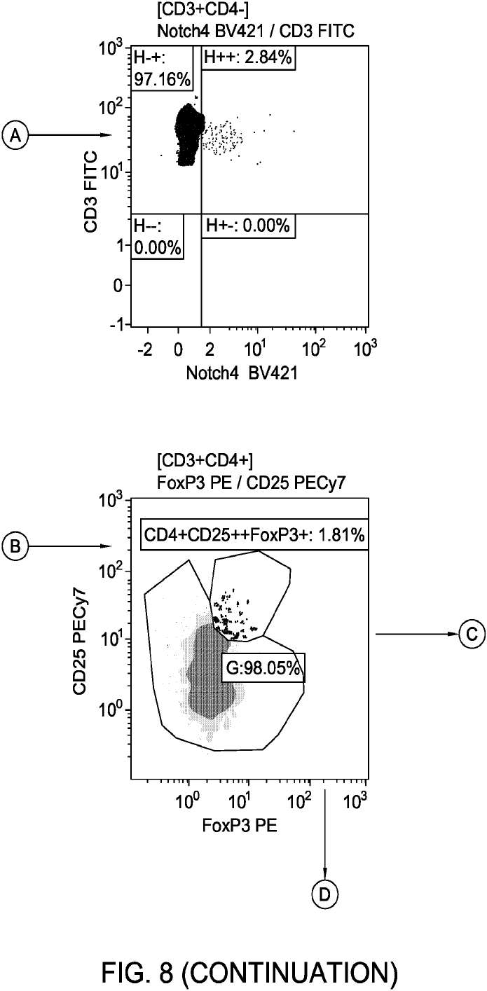

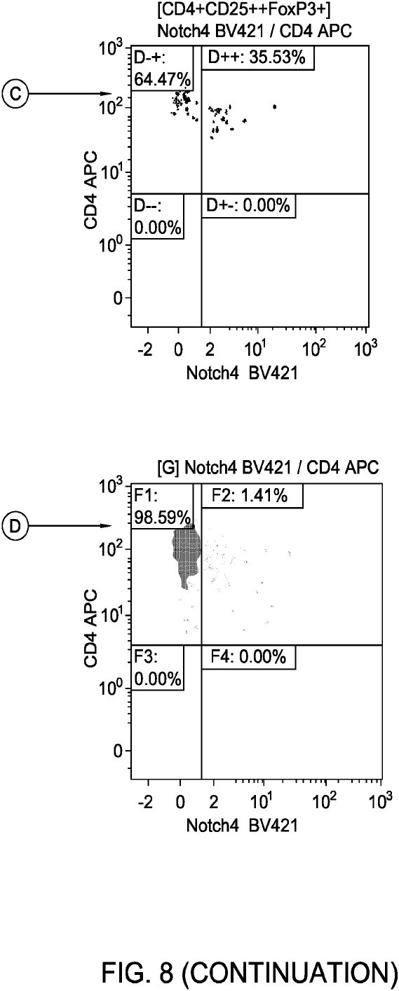

To establish the role of amphiregulin in the protection against lung inflammation induced by Notch4 depletion, the capacity of recombinant amphiregulin therapy to rescue Poly I:C-induced lung inflammation was examined. Treatment of Foxp3YFPCre mice with recombinant amphiregulin largely abrogated the weight loss and the inflammatory responses induced by Poly I:C treatment in association with the restoration of an intact epithelial barrier function (data not shown). Recombinant amphiregulin also reversed the skewing of lung macrophages towards an M1 phenotype upon in vitro treatment with Poly I:C (FIG. 7H), mirroring the same effect of Foxp3YFPCreNotch4Δ/Δ Treg cells (data not shown).

Consistent with the above results, analysis of the sera of COVID-19 subjects revealed increased amphiregulin concentrations in subjects with mild disease as compared to those of healthy controls, which declined in the sera of moderate and severe patients in a manner inversely proportional to Notch4 expression (data not shown). This inverse correlation between amphiregulin and Notch4 was particularly pronounced when comparing Notch4 and amphiregulin expression specifically in circulating Treg cells (data not shown). In contrast, there was no correlation between Notch4 and amphiregulin expression on circulating Teff cells (data not shown). Overall, these results are consistent with the suppression of amphiregulin production by Treg cells as a key mechanism of Notch4 inhibitory function in viral infections.

### Example 5

### Effect of Treg-Specific Notch4 Deletion in an H1N1 Influenza a Virus Infection Model

To extend these findings in vivo, the role of Notch4 expression on Treg cells in promoting lung tissue inflammation was analyzed in a mouse model of H1N1 Influenza A virus infection (Woodham et al., 2020). To sufficiently invoke an infection, mice were infected with H1N1 virus at 4×10⁴ plaque forming units (pfu)/mouse. Weight loss induced by the infection was abrogated upon Treg cell-specific deletion of Notch4. Analysis of Notch4 expression on long Treg cells revealed that H1N1 infection increased Notch4 expression, an effect abrogated by Treg cell-specific Notch4 deletion. Treg cell-specific Notch4 deletion greatly attenuated lung tissue neutrophil influx, restored the alveolar macrophage population, and reversed skewing of tissue macrophages away from the pro-inflammatory M1 phenotype toward an anti-inflammatory M2 phenotype.

TABLE 2

Demographics of COVID-19 patients stratified by disease severity and predictors of mortality

| | | Overall | Mild | Moderate | Severe | p-value |
|---|---|---|---|---|---|---|
| n | | 118 | 20 | 57 | 41 | |
| Site, n (%) | | | | | | <0.001 |
| Boston | | 38 (32.2) | 2 (10.0) | 17 (29.8) | 19 (46.3) | |
| Genoa | | 44 (37.3) | 0 (0.0) | 28 (49.1) | 16 (39.0) | |
| Istanbul | | 36 (30.5) | 18 (90.0) | 12 (21.1) | 6 (14.6) | |
| Age, mean (SD) | | 63.12 | 42.60 (15.22) | 65.21 (17.39) | 70.22 (15.83) | <0.001 |
| Male, n (%) | | 63 (53.4) | 9 (45.0) | 25 (43.9) | 29 (70.7) | 0.022 |
| White, n (%) | | 108 (91.5) | 18 (90.0) | 53 (93.0) | 37 (90.2) | 0.859 |
| BMI, mean (SD) | | 28.57 | 26.94 (3.94) | 27.98 (6.05) | 30.18 (8.69) | 0.151 |
| Any medical problems, | | 95 (81.2) | 8 (40.0) | 49 (86.0) | 38 (95.0) | <0.001 |
| Immunosuppression | | 16 (13.6) | 1 (5.0) | 6 (10.5) | 9 (22.0) | 0.125 |
| Malignancy | | 22 (18.6) | 1 (5.0) | 10 (17.5) | 11 (26.8) | 0.116 |
| Pulmonary disease | | 33 (28.0) | 2 (10.0) | 17 (29.8) | 14 (34.1) | 0.13 |
| Cardiac disease | | 70 (59.3) | 4 (20.0) | 32 (56.1) | 34 (82.9) | <0.001 |
| Hypertension | | 55 (46.6) | 4 (20.0) | 24 (42.1) | 27 (65.9) | 0.002 |
| Hyperlipidemia | | 12 (10.2) | 0 (0.0) | 6 (10.5) | 6 (14.6) | 0.205 |
| Endocrine disease | | 38 (32.5) | 2 (10.0) | 19 (33.3) | 17 (42.5) | 0.04 |
| Diabetes | | 26 (22.0) | 1 (5.0) | 14 (24.6) | 11 (26.8) | 0.126 |
| COVID-19 treatments | | | | | | |
| Remdesivir | | 26 (22.0) | 0 (0.0) | 8 (14.0) | 18 (43.9) | <0.001 |
| Hydroxychloroquine | | 54 (45.8) | 15 (75.0) | 24 (42.1) | 15 (36.6) | 0.014 |
| Glucocorticoids | | 67 (56.8) | 0 (0.0) | 35 (61.4) | 32 (78.0) | <0.001 |
| Anti-IL-6 | | 9 (7.6) | 0 (0.0) | 2 (3.5) | 7 (17.1) | 0.016 |
| Supportive care | | | | | | |
| Supplemental oxygen | | 89 (75.4) | 9 (45.0) | 50 (87.7) | 30 (73.2) | 0.001 |
| High flow oxygen | | 23 (19.5) | 0 (0.0) | 0 (0.0) | 23 (56.1) | <0.001 |
| Noninvasive ventilation | | 8 (6.8) | 0 (0.0) | 0 (0.0) | 8 (19.5) | <0.001 |
| Mechanical ventilation | | 20 (16.9) | 0 (0.0) | 0 (0.0) | 20 (48.8) | <0.001 |
| ECMO | | 2 (1.7) | 0 (0.0) | 0 (0.0) | 2 (4.9) | 0.148 |
| Dead | | 19 (16.1) | 0 (0.0) | 5 (8.8) | 14 (34.1) | <0.001 |
| 30-day, n (%) | | 8 (6.8) | 0 (0.0) | 0 (0.0) | 8 (19.5) | <0.001 |
| 90-day, n (%) | | 18 (15.3) | 0 (0.0) | 4 (7.0) | 14 (34.1) | <0.001 |
| Biomarkers, mean (SD) | | | | | | |
| Notch4, % | | 15.00 (14.48) | 4.67 (5.57) | 10.82 (10.93) | 25.84 (15.24) | <0.001 |
| IL-6, pg/mL | | 89.10 (209.25) | 124.22 (172.4 | 43.15 (38.47) | 137.70 | 0.071 |
| | Crude OR | 95% CI | p-value | Adjusted OR | 95% CI | P=value |
| Age, years | 1.086 | (1.044, 1.140) | <0.001 | 1.094 | (1.036, 1.177) | 0.005 |
| Male gender | 1.613 | (0.598, 4.654) | 0.354 | 3.713 | (0.942, 17.922) | 0.076 |
| History malignancy | 5.954 | (2.028, 17.718) | 0.001 | 3.717 | (0.950, 15.565) | 0.062 |
| Steroid treatment | 5.020 | (1.552, 22.55) | 0.015 | 5.730 | (1.177, 45.520) | 0.053 |
| Notch4, % | 1.052 | (1.020, 1.088) | 0.002 | 1.046 | (1.008, 1.090) | 0.023 |
| Interleukin-6, | 0.999 | (0.992, 1.001) | 0.553 | 1.000 | (0.993, 1.004) | 0.883 |

Disease severity classification. Mild = did not require hospitalization. Moderate = hospitalized but required only supplemental oxygen. Severe = hospitalized and required high flow oxygen, non-invasive ventilation, or mechanical ventilation.
Abbreviations: BMI = body mass index; ICU = intensive care unit; Anti-IL-6 = anti-interleukin-6 monoclonal antibody therapy; ECMO = extracorporeal membrane oxygenation; IL-6 = interleukin-6, OR = Odds Ratio. 95% CI = 95% confidence interval.

### Experimental Model and Subject Details for Examples 1-5

Human subjects. Three prospective cohorts of patients with COVID-19 from the following institutions: (1) Massachusetts General Hospital in Boston, Massachusetts, USA; (2) San Martino Hospital in Genoa, Italy; (3) Istanbul Medical Faculty Hospitals in Istanbul, Turkey were recruited. Inclusion criterion included a clinical syndrome consistent with COVID-19 disease and a positive SARS-CoV-2 test from a nasal swab based on RT-PCR. Severity of illness was defined as follows: (1) Mild for patients who did not require inpatient hospitalization; (2) Moderate for patients requiring inpatient hospitalization and who did not require therapies for acute respiratory failure such as high flow oxygen (defined as a flow rate of more than 15 liters per minute), non-invasive positive pressure ventilation, mechanical ventilation, and who did not require therapies for other types of organ failure such as renal replacement therapy or shock; (3) Severe for patients with organ failure requiring supportive therapies typically administered in the intensive care unit such as high flow oxygen, non-invasive positive pressure ventilation, mechanical ventilation, vasopressors, renal replacement therapy; (4) Convalescent for patients who have recovered from their acute illness and discharged from the hospital. A cohort of country-matched healthy controls were also recruited (Supplementary Data Table 1).

Clinical data including patient characteristics, therapies received, and clinical outcomes was abstracted from the medical record into a password-protected REDCap (Research Electronic Data Capture) database. A peripheral blood draw was obtained at the time of enrollment and weekly thereafter (if available) for flow cytometry and cytokine analysis.

Regulatory T-cell co-cultures with macrophages. Macrophages cells are isolated from lungs of Poly I:C-treated mice. Macrophages were isolated based on CD45⁺ F4/80⁺ MHCII⁺. Macrophages were then co-cultured (at 1:1 concentration) with lungs Treg cells from Foxp3^{YFPCre} or Foxp3^{YFPCre}Notch4^{Δ/Δ} mice Poly I:C-treated mice. Cells were treated with Poly I:C at a concentration of 10 ug/ml for three days. After 72 h, M1 (F4/80⁺ MHCII⁺ CD68⁺ CD80⁺CD86⁺) and M2 (F4/80⁺ MHCII⁺ CD163⁺ CD206⁺) polarization was measured by flow cytometric analysis.

Polyinosinic-polycytidylic acid (Poly I:C) mouse model. Mice were treated intratracheally with 2.5 mg/kg of Poly I:C HMW (InvivoGen) daily for six consecutive days. The weight of the mice was recorded daily upon application of the Poly I:C. The mice were subjected to airway hyperresponsiveness at day 7, then euthanized and analyzed. For blockading amphiregulin, mice were treated with a peptide spanning amino acids 91140 of the middle region of the human amphiregulin preproprotein (amphiregulin91-140 peptide; Mybiosource). The peptide was given intratracheally at 10 µg/ml in PBS in a final volume of 100 µl. For the amphiregulin neutralizing antibody tests, the mice were given intraperitoneally 20 µg of goat anti-mouse amphiregulin mAb (clone AF989; R&D systems) or isotype control mAb (clone AB-108-C; R&D systems) daily for the duration of the experiment.

H1N1 influenza A virus preparation. Mouse-adapted H1N1 Influenza A virus (PR/8/34) was obtained from Charles River (Catalogue no. 10100374). Viral stocks were calculated to contain 40,000 infectious units (IU) per mouse and were diluted to a volume of 20 µl/mouse in PBS. For lethal dose (LD75), the viral stocks were calculated to contain 60,000 IU per mouse and diluted to a volume of 20 µl/mouse in PBS as well.

H1N1 Influenza A viral infection model. Mice were treated intranasally on day 0 of the experiments with either a 40,000 pfu dose of the H1N1 virus or 70,000pfu dose, equivalent to a lethal dose 75 (LD75), as indicated. The mice were monitored on a daily basis to see signs of infection. The weights of the mice were recorded and once a mouse weight loss exceeded 20-25%, the mouse was euthanized. The endpoint of the experiments were set at day 12, to capture the peak of inflammation.

M1 and M2 gating strategy. M1 was defined as follows: CD45⁺CD4⁻F4/80⁺MHCII⁺ CD68⁺ CD80⁺CD86⁺ while M2 macrophages were defined as follows: CD45⁺CD4⁻F4/80⁺ MHCII+CD163+CD206+.

Statistical analysis. Student’s two-tailed t-test, one- and two-way ANOVA and repeat measures two-way ANOVA with post-test analysis and log-rank test of groups were used to compare test groups, as indicated. Linear Regression was used for correlation analysis. For analysis of the human data, summary statistics were calculated using number (percentage) for binary and categorical data and mean (standard deviation) or median (interquartile range) for continuous data depending on the normality of the distribution. Logistic regression was performed to determine the association between Notch4 expression and mortality. Covariates including age, gender, and study site were selected based on a priori knowledge. In the case of highly correlated covariates, we chose the predictor that had the strongest univariate association between the potential predictor and outcome. In the final model, the outcome was death at any time after study enrollment, the predictors of interest were Notch4 expression and serum interleukin-6 levels, and covariates included age, gender, history of malignancy, and corticosteroid treatment. Analyses were performed in R version 3.6.1. Two-sided p-values of <0.05 were considered statistically significant.

### Example 6: Notch4 Modulation in an H1N1 Influenza Mouse Model

In this example, the role of Notch4 expression on Treg cells in promoting lung tissue inflammation is analyzed in a mouse model of H1N1 Influenza A virus infection (see, e.g., Woodham et al., 2020).

To sufficiently invoke an infection, mice are infected with H1N1 virus at 4×10⁴ plaque forming units (pfu)/mouse. To determine if a Notch4 modulating agent (e.g., a humanized Notch4 antibody) can be protective of an H1N1 viral infection, mice are treated with a Notch4 modulating agent prior to the onset of an infection; e.g., prior to infection with the virus, or prior to a detectable H1N1 infection in the mouse following infection with the virus. Alternatively, to determine if a Notch4 modulating agent can rescue an H1N1 viral infection, mice are treated with a Notch4 modulating agent following the onset of an infection.

To assess the efficacy of a Notch4 modulating agent in protecting and/or rescuing a viral infection, various symptoms of the infection are assessed. For example, an H1N1 infection in the mouse will induce weight loss, increase BAL fluid IL-6 concentrations, and induce a lung tissue inflammatory response. Hallmarks of a lung tissue inflammatory response include lung tissue neutrophil influx, depletion of the alveolar macrophage population, and skewing of tissue macrophages away from the pro-inflammatory M1 phenotype towards an anti-inflammatory M2 phenotype. Further, Notch4 expression on lung Treg cells is increased in the mouse following onset of infection.

Prevention of the onset of the symptoms noted above by administration of a Notch4 modulating agent prior to infection with the virus would indicate that the agent is protective against the viral infection.

Reversal of the onset of the symptoms noted above by administration of a Notch4 modulating agent post infection (e.g., at days 4 to 7 post infection) would indicate that the agent rescues, or treats, the viral infection.

These experiments are further performed in a humanized immune system mouse model, e.g., NOD- Prkdc^{scid}Il2rg^{tmiwjl}/Sz (NSG) mice reconstituted with human PBMCs.

### Materials and Methods

H1N1 influenza A virus preparation may be performed substantially as described in Example 5. An H1N1 Influenza A viral infection model may be generated substantially as described in Example 5. (See Materials and Methods section for Examples 1-5).

Humanized mice H1N1 viral infection. NOD-Prkdc^{scid}Il2rg^{tmiw}j¹/Sz (NSG) humanized mice are reconstituted with PBMCs from healthy control. Then, mice are treated with a sublethal dose of the virus as indicated intranasally on day 0. The mice are monitored on a daily basis to see signs of infection. The weights of the mice are recorded and once a mouse weight loss exceeded 20-25%, the mouse is euthanized. For sublethal experiments, the endpoint of the experiment is set at day 12, to capture the peak of inflammation.

### Example 7: Notch4 Modulation in a Mouse Injurious Mechanical Ventilation Model of Acute Respiratory Distress Syndrome

In this example, a Notch4 modulating agent is tested in an injurious mechanical ventilation model of acute respiratory distress syndrome.

Mice are pretreated with varying doses of a Notch4 modulating agent (such as a humanized Notch4 antibody) or saline and receive either protective (8 mL/kg) or injurious (25 mL/kg) ventilation for four hours. The Notch4 modulating agent or saline is injected intraperitoneally at days -2, -1, and 0 of the experiment.

Lung mechanics (e.g., respiratory system elastance, tissue damping, and airway resistance) are evaluated by forced oscillation technique. Respiratory system compliance is measured with quasi-static pressure-volume curves.

Hematoxylin-eosin-stained lung sections are scored for the presence of lung injury. Pulmonary endothelial dysfunction is ascertained by bronchoalveolar lavage protein content and lung tissue expression of endothelial junctional protein Vascular Endothelial cadherin by immunoblotting. To assess the inflammatory response in the lung, bronchoalveolar lavage fluid total cell content and neutrophil fraction is assessed by microscopy and staining, as well as enzyme-linked immunosorbent assay (ELISA) for Matrix-Metalloprotease-9. To evaluate the systemic response, plasma levels of Tumor Necrosis Factor-α, Interleukin-6, and Matrix-Metalloprotease-9 are determined by ELISA.

Any or any combination of the following observations may indicate a therapeutic effect of the Notch4 modulating agent: reduction of lung mechanical alterations induced by ventilation with high tidal volume, lower histologic lung injury score, attenuation of lavage pleocytosis and neutrophilia, lower microvascular protein permeability, lower Tumor Necrosis Factor-α levels, lower plasma Interleukin-6 levels, and lower lavage fluid Matrix-Metalloproteinase-9 levels, and preserved or higher levels of lung tissue vascular endothelial cadherin expression relative to saline controls.

### Example 8: Notch4 Modulation in a Pig Model of Endotoxin-Induced Acute Respiratory Distress Model

In this example, a Notch4 modulating agent is tested in an endotoxin-induced model of acute respiratory distress syndrome.

Pigs are anesthetized, intubated, surgically instrumented for hemodynamic monitoring, and randomized into three groups of similar sizes: (1) control (surgical instrumentation only); (2) lipopolysaccharide (LPS) (infusion of Escherischia coli lipopolysaccharide at 100 µg/kgl and (3) Notch4 modulating agent (e.g., a humanized Notch4 antibody) + LPS. Additional groups may be used to test different doses of Notch4 modulating agent. Group (3) and possible additional groups are administered Notch4 modulating agent 12 hours before LPS infusion.

Animals are monitored for 6 h following LPS or sham LPS infusion. Serial bronchoalveolar lavage (BAL) samples are analyzed for matrix metalloproteinase concentration by gelatin zymography. Lung tissue is fixed for morphometric assessment at necropsy.

LPS infusion is marked by significant physiological deterioration as compared to the control group, including increased plateau airway pressure (Pplat) and a decrement in arterial oxygen partial pressure (PaO2). Reduction of the above pathophysiological changes after LPS infusion in the Notch4 modulating agent + LPS group(s) may indicate that the Notch4 modulating agent has a therapeutic effect.

MMP-9 and MMP-2 concentration in BAL fluid is typically significantly increased after LPS infusion. Reduction of increase in MMP-9 and MMP-2 concentrations by pretreatment of a Notch4 modulating agent may indicate that the Notch4 modulating agent has a therapeutic effect.

Morphometrically, LPS causes a significant sequestration of neutrophils and monocytes into pulmonary tissue. Amelioration of this response by pretreatment with a Notch4 modulating agent may indicate that the Notch4 modulating agent has a therapeutic effect.

### Example 9: Treatment of Human Subjects With a Notch4 Modulator

A humanized Notch4 antibody is administered to a human suffering or at risk from suffering from acute respiratory distress syndrome in an amount effective to treat acute respiratory distress syndrome.

1) A method for treating or ameliorating a symptom associated with a coronavirus infectious disease, the method comprising administering to a subject having a coronavirus infectious disease an effective amount of an agent that inhibits Notch4.

2) The method of claim 1, wherein the coronavirus infectious disease is COVID-19.

3) The method of claim 1, further comprising the step of, prior to administering, diagnosing the subject as having coronavirus infectious disease.

4) The method of claim 1, further comprising the step of, prior to administering, receiving the results of an assay that diagnoses the subject as having coronavirus infectious disease.

5) The method of claim 1, wherein the agent that inhibits Notch4 is selected from the group consisting of a small molecule, an antibody or antigen-binding fragment thereof, a peptide, a genome editing system, an antisense oligonucleotide, and an RNAi.

6) The method of claim 5, wherein the antibody or antigen-binding fragment thereof is a humanized antibody or antigen-binding fragment thereof.

7) The method of claim 5, wherein the RNAi is a microRNA, an siRNA, or a shRNA.

8) The method of any one of claims 1-7, wherein inhibiting Notch4 is inhibiting the expression level and/or activity of Notch4.

9) The method of claim 8, wherein the expression level and/or activity of Notch4 is inhibited by at least 50%, at least 60%, at least 70%, at least 80%, at least 90%, or more as compared to an appropriate control or reference level.

10) The method of claim 1, wherein Notch4 is inhibited on T regulatory cells.

11) The method of claims 1, further comprising administering at least one additional therapeutic.

12) The method of claim 11, wherein the at least one additional therapeutic is an anti-viral therapeutic.

13) A method for treating or ameliorating a symptom of COVID-19, the method comprising administering to a subject having COVID-19 an effective amount of an agent that inhibits Notch4.

14) The method of claim 13, further comprising the step of, prior to administering, diagnosing the subject as having COVID-19.

15) The method of claim 13, further comprising the step of, prior to administering, receiving the results of an assay that diagnoses the subject as having COVID-19.

16) A method for preventing a coronavirus infectious disease, comprising administering to a subject at risk of developing a coronavirus infectious disease an agent that inhibits Notch4.

17) The method of claim 16, further comprising the step of, prior to administering, identifying a subject at risk of developing coronavirus infectious disease.

18) The method of claim 16, further comprising the step of, prior to administering, receiving the results of an assay that identifies a subject as being at risk of developing coronavirus infectious disease.

19) The method of claim 16, wherein the coronavirus infectious disease is COVID-19.

20) A method for preventing COVID-19, comprising administering to a subject at risk of developing a COVID-19 disease an agent that inhibits Notch4.

21) The method of claim 20, further comprising the step of, prior to administering, identifying a subject at risk of developing COVID-19.

22) The method of claim 20, further comprising the step of, prior to administering, receiving the results of an assay that identifies a subject as being at risk of developing COVID-19.

23) A composition for the treatment of a coronavirus infectious disease, the composition comprising an agent that inhibits Notch4 and a pharmaceutically acceptable carrier.

24) A composition for the treatment of a COVID-19, the composition comprising an agent that inhibits Notch4 and a pharmaceutically acceptable carrier.

25) The composition of claims 23 or 24, wherein the agent that inhibits Notch4 is selected from the group consisting of a small molecule, an antibody or antigen-binding fragment thereof, a peptide, a genome editing system, an antisense oligonucleotide, and an RNAi.

26) The composition of claims 23 or 24, wherein the antibody or antigen-binding fragment thereof is a humanized antibody or antigen-binding fragment thereof.

27) The composition of claims 23 or 24, wherein the RNAi is a microRNA, an siRNA, or a shRNA.

28) The composition of claims 23 or 24, wherein the composition is formulated for inhaled administration.

29) A method for treating a subject at risk of developing acute respiratory distress syndrome (ARDS), the method comprising,
a. receiving the results of an assay that identifies a subject as being at risk of developing ARDS when the level of Notch is increased as compared to a reference level; and
b. administering an agent that inhibits Notch4 to a subject identified as being at risk of developing ARDS.

30) The method of claim 29, wherein the subject was diagnosed as having COVID-19 prior to obtaining a biological sample.

31) The method of claim 29, further comprising the step of, prior to obtaining a biological sample, diagnosing a subject as having COVID-19.

32) The method of claim 29, further comprising the step of, prior to obtaining a biological sample, receiving the results of an assay that diagnoses a subject as having COVID-19.

33) The method of claims 29, wherein the level of Notch4 is increased by at least 2-fold, at least 3-fold, at least 4-fold, at least 5-fold, at least 6-fold, at least 7-fold, at least 8-fold, at least 9-fold, at least 10-fold, or more as compared to a reference level.

34) A method for treating a subject having acute respiratory distress syndrome (ARDS) or ameliorating a symptom associated with ARDS, the method comprising,
a. receiving the results of an assay that identifies a subject as having ARDS when the level of Notch is increased as compared to a reference level; and
b. administering an agent that inhibits Notch4 to a subject identified as having ARDS.

35) The method of claims 34, wherein the level of Notch4 is increased by at least 2-fold, at least 3-fold, at least 4-fold, at least 5-fold, at least 6-fold, at least 7-fold, at least 8-fold, at least 9-fold, at least 10-fold, or more as compared to a reference level.

36) A method for treating, preventing, or ameliorating a symptom associated with a coronavirus infectious disease, the method comprising administering to a subject having or at risk of having a coronavirus infectious disease an effective amount of a Notch4 modulating agent.

37) The method of claim 36, wherein the coronavirus infectious disease is COVID19.

38) The method of claim 36 or 37, wherein the Notch4 modulating agent is selected from the group consisting of a small molecule, an antibody or antigen-binding fragment thereof, a peptide, a genome editing system, an antisense oligonucleotide, and an RNAi.

39) The method of claim 38, wherein the Notch4 modulating agent is a Notch4 antibody or Notch4-binding fragment thereof.

40) The method of claim 39, wherein the Notch4 antibody is a humanized antibody or Notch4-binding fragment thereof.

41) The method of any one of claims 36-40, wherein the Notch4 modulating agent reduces the expression level and/or activity of Notch4.

42) The method of claim 41, wherein the Notch4 modulating agent reduces the expression level and/or activity of Notch4 by at least 50%, at least 60%, at least 70%, at least 80%, at least 90%, or more as compared to an appropriate control or reference level.

43) A composition for the treatment, prevention, or amelioration of a symptom associated with coronavirus infectious disease, the composition comprising a Notch4 modulating agent and a pharmaceutically acceptable carrier.

44) A method of treating or preventing acute respiratory distress syndrome (ARDS), in a subject in need thereof, the method comprising,
administering to the subject an effective amount of an agent that modulates Notch4.

45) The method of claim 44, wherein the agent that modulates Notch4 is selected from the group consisting of a small molecule, an antibody or antigen-binding fragment thereof, a peptide, a genome editing system, an antisense oligonucleotide, and an agent that is an RNA interfering (RNAi) agent.

46) The method of claim 45, wherein the antibody or antigen-binding fragment thereof is a humanized antibody or antigen-binding fragment thereof.

47) The method of claim 46, wherein the antibody or antigen-binding fragment thereof is a humanized Notch4 antibody or antigen-binding fragment thereof.

48) The method of claim 45, wherein the RNAi agent is a microRNA, an siRNA, or a shRNA.

49) The method of claims 44-48, wherein Notch4 is modulated by reducing the expression level and/or activity of Notch4.

50) The method of claim 49, wherein the expression level and/or activity of Notch4 is reduced by at least 50%, at least 60%, at least 70%, at least 80%, at least 90%, or more as compared to an appropriate control or reference level.

51) The method of any one of claims 44-50, wherein the agent modulates Notch4 by inhibiting expression and/or activity of Notch4.

52) A method of treating or preventing acute respiratory distress syndrome (ARDS), in a subject in need thereof, the method comprising,
administering to the subject an effective amount of an antibody or antigen-binding fragment thereof that modulates Notch4 activity.

53) The method of claim 52, wherein the antibody or antigen-binding fragment thereof is a humanized antibody, a chimeric antibody, a nanobody, an affibody, an scFv, an Fab, or a antigen-binding fragment thereof.

54) The method of any one of claims 44-53, wherein Notch4 is modulated on T regulatory cells.

55) The method of any one of claims 44-53, further comprising administering at least one additional therapeutic.

56) The method of claim 55, wherein the at least one additional therapeutic is an anti-viral therapeutic.

57) The method of any one of claims 43-56, wherein, before the step of administering, the level of Notch4 in the subject is increased by at least 2-fold, at least 3-fold, at least 4-fold, at least 5-fold, at least 6-fold, at least 7-fold, at least 8-fold, at least 9-fold, at least 10-fold, or more as compared to a reference level.

58) The method of claim 57, wherein the level of Notch4 in the subject is determined before administration.

59) The method of claim 58, wherein the level of Notch4 in the subject is monitored after administration of the agent.
